(19) United States
(12) Patent Application Publication
(10) Pub. No.: US 2026/0159840 A1
(43) Pub. Date: Jun. 11, 2026

(54) METHODS OF TREATING IMPAIRED GLUCOCORTICOID SIGNALLING IN UTERINE DISORDERS

(71) Applicant: UNIVERSITY OF SOUTH FLORIDA, Tampa, FL (US)

(72) Inventors: Charles Joseph Lockwood, Tampa, FL (US); Ozlem Guzeloglu-Kayisli, Tampa, FL (US); Carrie Ann Malcom, Tampa, FL (US); Umit Ali Kayisli, Tampa, FL (US)

(21) Appl. No.: 19/412,013

(22) Filed: Dec. 8, 2025

Related U.S. Application Data
(60) Provisional application No. 63/728,910, filed on Dec. 6, 2024.

(51) Int. Cl. C12N 15/113; A61P 35/00; C12Q 1/6883

(52) U.S. Cl. CPC C12N 15/1137; A61P 35/00; C12Q 1/6883; C12Y 502/01008; C12N 2310/11; C12N 2310/14; C12Q 2600/158

(57) ABSTRACT

The present disclosure relates to methods of modulating FK506-binding protein-51 (FKBP51) mediated glucocorticoid signalling and HSD11B1 expression in uterine disorders. Further disclosed are compositions comprising FKBP5 gene modulating agents and use thereof.

### CROSS-REFERENCE TO RELATED APPLICATIONS

This application claims the benefit of priority to U.S. Provisional Ser. No. 63/728,910, filed on Dec. 6, 2024, the disclosure of which is expressly incorporated by reference herein in its entirety.

### TECHNICAL FIELD

The present disclosure provides compositions and methods for modulating FKBP51-mediated glucocorticoid signalling axis for diagnostic, prognostic, and therapeutic purposes in uterine disorders.

### BACKGROUND

Uterine leiomyomata, also known as uterine fibroids, are the most common benign gynecologic tumors, occurring in 50% to 70% of females by menopause, with rates reaching over 80% in Black women. Leiomyomata originate from uterine smooth muscle cells in the myometrium, and their growth is primarily stimulated by ovarian steroids estrogen and progesterone. Uterine fibroids are classically diagnosed with physical exam and ultrasound imaging, which is highly sensitive for detecting this pathology. Current therapies typically provide only short-term relief and the cross-over rate to surgery is high. While many leiomyomata are asymptomatic and discovered incidentally, ˜25% to 30% of women experience a spectrum of symptoms that increase morbidity and adversely affect their quality of life. The most common symptoms include abnormal uterine bleeding (AUB), heavy menstrual bleeding (HMB), pelvic pain and pressure, low back pain, anemia, and bladder and bowel dysfunction. They continue to be the leading indication for hysterectomy. Additionally, the presence of one or multiple leiomyomata may affect fertility, as the distortion of the uterus can prevent successful implantation and the continued survival of an intrauterine pregnancy. Uterine fibroids also increase the risk of certain obstetric complications, including recurrent pregnancy loss (RPL), preterm labor (PTL), abnormal placentation, the need for cesarean birth, and postpartum hemorrhage.

The healthy endometrium regenerates approximately 450 times in a women's reproductive life without the formation of scar tissue. This process of endometrium regeneration is orchestrated, in part, by an endometrial progenitor cell population. Deregulation of this cycle is seen in endometrial pathologies such as Asherman's syndrome (AS), where patients can suffer from endometrial scarring, fibrosis, and adhesions occluding the uterine cavity. Endometrial research to date has primarily focused on factors regulating embryo implantation, emphasizing the importance of decidualization and placentation for fertility and pregnancy success. Understanding of the endometrial stromal compartment's role in regeneration after menstrual shedding remains scarce. Further research is critical in developing a better biological understanding of benign gynecological diseases such as endometriosis, and leiomyomas, with the aim of developing suitable therapies. Cell therapy has been a steadily expanding field with 327 active clinical trials so far, involving “mesenchymal cells.” These cell products have been primarily developed to alleviate diseases with pro-inflammatory components, or a need to repair damaged tissue. There is a critical need for new therapeutic interventions that selectively target the pathways involved in uterine fibroid growth. Estrogen and progesterone are established drivers of uterine fibroid proliferation; yet, some women continue to experience symptomatic uterine fibroids following menopause, suggesting the presence of other contributing factors.

With the current trend of increasing age at childbearing, understanding fertility-sparing management of uterine leiomyomata is critical; therefore, the identification of a novel signaling pathway that regulates uterine fibroid growth through local regulation may aid in the development of new, targeted medical therapeutics.

### SUMMARY

Disclosed herein are methods and compositions of treating, inhibiting, reducing, decreasing, ameliorating, and/or preventing uterine disorders with impaired glucocorticoid signalling in a subject in need thereof.

In one example, disclosed herein is a method of treating a uterine disorder in a subject, comprising:
determining an expression score of one or more biomarkers in a biological sample obtained from a subject relative to a reference control,
   wherein the one or more biomarkers comprise one or more RNAs, or an expression product thereof, of genes selected from the group consisting of TSC22D3, ADH1B, IL1R1, HSD11B1, GREM1, IGFBP5, AMIGO2, and FKBP5;
determining a cumulative expression score from the one or more expression scores of the one or more biomarkers; and
administering to the subject a therapeutically effective amount of an FKBP5-modulating agent if the cumulative expression score is lower than a threshold.

In some examples, the expression score greater than 1 indicates upregulation relative to the reference control and an expression score less than 1 indicates downregulation relative to the reference control.

In some examples, the FKBP5 modulating agent comprises an FKBP5 small interfering RNA (siRNA).

In some examples, the FKBP5 modulating agent comprises a small molecule inhibitor of FKBP51 protein activity.

In some examples, the FKBP5 modulating agent comprises an inhibitor of HSD11B1.

In some examples, the biological sample comprises leiomyoma tissue, paired myometrial tissue, myometrial tissue, endometrial stromal cells, or any combination thereof.

In some examples, the uterine disorders comprise uterine leiomyoma, uterine fibroids, abnormal uterine bleeding, myometrial hyperplasia, progesterone resistant uterine disorder, estrogen responsive uterine disorder, endometriosis, pelvic pain, benign adnexal mass, adenomyosis, uterine cancer, polycystic ovary syndrome (PCOS) or cervical dysplasia.

In one example, disclosed herein is a method of treating a uterine disorder in a subject, comprising:
determining an expression score of a first group of one or more biomarkers, a second group of one or more biomarkers, a third group of one or more biomarkers, and a fourth group of one or more biomarkers in a biological sample obtained from a subject relative to a reference control,
wherein the first group of one or more biomarkers comprise one or more RNAs, or an expression product thereof, of the genes selected from the group consisting of CNN1,MYH9, MYH10, and ACTA2, the second group of one or more biomarkers comprise one or more RNAs, or an expression product thereof, of genes selected from the group consisting of GREM1, IGFBP5, and AMIGO2, the third group of one or more biomarkers comprise one or more RNAs, or an expression product thereof, of the genes selected from the group consisting of LAMA2, LAMB1, and FN1, and the fourth group of one or more biomarkers comprise one or more RNAs, or an expression product thereof, of the genes selected from the group consisting of TSC22D3, ADH1B, IL1R1, and HSD11B1;
determining a first expression score by integrating the expression scores of the one or more biomarkers in the first group, a second expression score by integrating the expression score of the one or more biomarkers in the second group, a third expression score by integrating the expression score of the one or more biomarkers in the third group, and a fourth expression score by integrating the expression score of the one or more biomarkers in the fourth group; and
administering to the subject a therapeutically effective amount of an FKBP5 modulating agent if the first expression score is lower than a first threshold, the second expression score is lower than a second threshold, the third expression score is higher than a third threshold, and the fourth expression score is higher than a fourth threshold.

In some examples, wherein the therapeutically effective amount of the FKBP5 modulating agent is administered to the subject intravenously, intramuscularly, intraperitoneally, intradermally, or subcutaneously. In some examples, the therapeutically effective amount is administered as at least a single dose or multiple doses, wherein a single dose is administered at a concentration of at least 0.005 mg/kg/day to 30 mg/kg/day, or at least from 0.01 mg/kg/day to 10 mg/kg/day, or from 0.01 mg/kg/day to 2 mg/kg/day.

Some examples comprise administering to the subject a therapeutically effective amount of the FKBP5 modulating agent if the first expression score is lower than 0.5, the second expression score is lower than 0.5, the third expression score is higher than 2, and the fourth expression score is higher than 2.

In some examples, the biological sample from the subject has a higher expression of FKBP51 or HSD11β1 protein as compared to the reference control.

In some examples, the FKBP5 modulating agent comprises an FKBP5 small interfering RNA (siRNA). In some examples, the siRNA is administered in combination with at least one anti-angiogenic agent, an anti-tumor agent, an immunotherapeutic agent, or a combination thereof.

In some examples, the FKBP5 modulating agent comprises a small molecule inhibitor of FKBP51 protein activity. In some examples, the FKBP5 modulating agent comprises an oligonucleotide, an antisense oligonucleotide, a DNA oligonucleotide, an RNA oligonucleotide, an RNA oligonucleotide having at least a portion of said RNA oligonucleotide capable of hybridizing with RNA to form an oligonucleotide-RNA duplex, or a chimeric oligonucleotide. In some examples, the FKBP5 antisense oligonucleotide comprises at least one modified internucleoside linkage, sugar moiety, or nucleobase. In some examples, the FKBP5 antisense oligonucleotide comprises at least one 2′-O-methoxyethyl sugar moiety. In some examples, the FKBP5 antisense oligonucleotide comprises at least one phosphorothioate internucleoside linkage. In some examples, the FKBP5 antisense oligonucleotide comprises at least one 5-methylcytosine.

In some examples, the FKBP5 modulating agent comprises an inhibitor of HSD11B1.

In some examples, the biological sample comprises leiomyoma tissue, paired myometrial tissue, myometrial tissue, endometrial stromal cells, or any combination thereof.

In some examples, the uterine disorders comprise uterine leiomyoma, uterine fibroids, abnormal uterine bleeding, myometrial hyperplasia, progesterone resistant uterine disorder, estrogen responsive uterine disorder, endometriosis, pelvic pain, benign adnexal mass, adenomyosis, uterine cancer, polycystic ovary syndrome (PCOS) or cervical dysplasia.

In one example, disclosed herein is a method of inhibiting the expression of FKBP5 gene or HSD11B1 gene in cells or tissues comprising contacting said cells or tissues with an inhibitor or modulating agent comprising antisense oligonucleotides or a pharmaceutically effective composition to inhibit the expression of FKBP51 or HSD11B1.

In one example, disclosed herein is a method of screening for a FKBP5 modulating agent, wherein the method comprises: contacting a preferred target segment of a nucleic acid molecule encoding FKBP5 gene with one or more candidate modulators of FKBP5 gene, and identifying one or more modulators of FKBP5 gene expression which modulate the expression of FKBP5 gene.

In one example, disclosed herein is a pharmaceutical composition, comprising: a FKBP5 modulating agent; and a therapeutically acceptable carrier.

In some examples, the FKBP5 modulating agent comprises an FKBP5 small interfering RNA (siRNA).

In some examples, the FKBP5 siRNA comprises 8-30 nucleotides targeting a nucleic acid molecule encoding FKBP5 gene.

In some examples, the FKBP5 modulating agent comprises a small molecule inhibitor of FKBP51 protein activity.

In some examples, the FKBP5 modulating agent comprises an inhibitor of HSD11B1.

In some examples, the therapeutically acceptable carrier comprises polymer micelle, liposome, emulsion, microsphere, or nanosphere.

In some examples, the FKBP5 modulating agent is an antisense oligonucleotide, wherein the antisense oligonucleotide is a DNA oligonucleotide, an RNA oligonucleotide, or a chimeric oligonucleotide.

In some examples, the antisense oligonucleotide hybridizes with an FKBP5 RNA to form an oligonucleotide-RNA duplex.

In some examples, the FKBP5 modulating agent shares at least 70% complementarity with a nucleic acid molecule encoding FKBP5 gene. In some examples, the FKBP5 modulating agent shares at least 80% complementarity with a nucleic acid molecule encoding FKBP5 gene. In some examples, the FKBP5 modulating agent shares at least 90% complementarity with a nucleic acid molecule encoding FKBP5 gene. In some examples, the FKBP5 modulating agent shares at least 95% complementarity with a nucleic acid molecule encoding FKBP5 gene.

Uterine fibroids are highly prevalent in reproductive age women and have significant morbidity. Moreover, uterine fibroids represent a considerable healthcare burden, as non-surgical therapies typically provide only short-term relief and the cross-over rate to surgery is high. There is a critical need for new therapeutic interventions that selectively target the pathways involved in uterine fibroid growth. Estrogen and progesterone are established drivers of uterine fibroid proliferation; yet, some women continue to experience symptomatic uterine fibroids following menopause, suggesting the presence of other contributing factors. The current disclosure unveils the potential of FKBP51 co-chaperone in glucocorticoid receptor signaling and treatment/prevention of uterine disorders. Glucocorticoids are strong transcriptional regulators in uterine fibroid cells and can induce uterine fibroid cell proliferation. However, it is unclear whether glucocorticoids are a biologically active signaling pathway in vivo, as the glucocorticoid activating enzyme, 11βHSD-1, has never been studied in the context of uterine fibroids. The purpose of this invention is to measure the presence of 11βHSD-1 in uterine fibroid tissue and determine that this enzyme functionally activate glucocorticoids in primary uterine fibroid cells.

### BRIEF DESCRIPTION OF THE FIGURES

The accompanying figures, which are incorporated in and constitute a part of this specification, illustrate several examples described below.

FIG. 1A and FIG. 1B show that FKBP5-silencing reduces dexamethasone-induced differentially regulated genes in control-silencing leiomyoma cells. (FIG. 1A) Volcano plots of RNA-sequencing data (n=4 each group) comparing control leiomyoma cells (C) or cells transfected with FKBP5 siRNA (FK) with dexamethasone (DEX)-treated cells (C-DEX vs C or FKD-DEX vs FK) show differentially up-or down-regulated genes expressed as log2-fold change. (FIG. 1B) Venn diagrams display the specific number of genes that are upregulated and downregulated in DEX-treated control or FKBP5 knockdown cells vs control or FKBP5 knockdown cells, respectively; and which genes are uniquely up-or down-regulated or common between the 2 groups. RNA sequencing raw data available in the NCBI GEO repository, accession number GSE292403.

FIG. 2A, FIG. 2B, and FIG. 2C show the FKBP5 knockdown in leiomyoma cells treated with dexamethasone predicts inhibition of glucocorticoid signaling. (FIG. 2A) Volcano plot of RNA-sequencing data (n=4 each group) directly compares differential gene expression in dexamethasone (DEX)-treated FKBP5-silenced (FK-DEX) vs control (C-DEX) leiomyoma cells. (FIG. 2B) Heat map depicts the activation Z-score for RNA-sequencing data, comparing DEX-treated control (Csi-Dex) vs vehicle-treated control leiomyoma cells (Csi), DEX-treated FKBP5-silenced (FKsi-Dex) vs vehicle-treated FKBP5-silenced leiomyoma cells (FKsi), and DEX-treated FKBP5-silenced vs DEX-treated control cells (FKsi-Dex vs Csi-Dex). Silencing of FKBP5 results in the inhibition of dexamethasone and glucocorticoid receptor (NR3C1) downstream signaling. (FIG. 2C) Ingenuity Pathway-Upstream Regulator Analysis indicates that the upstream target NR3C1 is predicted to be inhibited in DEX-treated FKBP5-silenced vs DEX-treated control (FKsi-Dex vs Csi-Dex) leiomyoma cells. RNA sequencing raw data available in the NCBI GEO repository, accession number GSE292403.

FIG. 3A and FIG. 3B show silencing of FKBP5 blunts dexamethasone (DEX)-regulated expression of glucocorticoid response element-containing genes in primary leiomyoma cell cultures. qPCR analysis of mRNA levels for DEX-upregulated genes TSC22D3, ADH1B, IL1R1 (FIG. 3A), and DEX-downregulated genes GREM1, IGFBP5, AMIGO2 (FIG. 3B) in cultured leiomyoma cells transfected with either control or FKBP5 siRNA for 48 hours and then treated with either vehicle (Cont) or DEX for 24 hours. Bars represent mean ±standard error of the mean, n=4.

FIG. 4A, FIG. 4B, FIG. 4C, and FIG. 4D show that FKBP51 promotes increased HSD11B1 expression, with baseline HSD11B1 levels elevated in leiomyomatous uteri. HSD11B1 levels in leiomyoma cells (n=4) (FIG. 4A) by RNA-sequencing representing fragments per kilobase of transcript per million mapped reads (FPKM) and (FIG. 4B) by qPCR. Dexamethasone (DEX) treatment increased HSD11B1 mRNA levels in control siRNA-transfected cells vs vehicle treatment (Cont). However, this DEX-induced upregulation was blunted in FKBP5-silenced cells. (FIG. 4C) In situ HSD11B1 mRNA levels are increased in paired leiomyoma tissue samples (n=17 paired-myometrial, n =17 leiomyoma) relative to normal myometrium samples (n=16). (FIG. 4D) HSD11β1 immunostaining (brown) in normal myometrium (n=6), paired myometrial (n=6) and leiomyoma (n=6) tissue samples, with decreased expression seen in normal tissues as quantified by H score. Inset represents negative control in each tissue sample. Bars represent mean ±standard error of the mean. *P<0.05, *P<0.001, **P=0.002, **P=0.01, *P=0.04, RNA sequencing raw data available in the NCBI GEO repository, accession number GSE292403.

FIG. 5A and FIG. 5B show that dexamethasone (DEX) treatment increases HSD11B1 expression in normal myometrial and human endometrial stromal cell cultures but to a lesser extent than in leiomyoma cells, in a FKBP51-dependent manner. (FIG. 5A) FKBP5 knockdown efficiency in normal myometrial cell (TM; n=4) and human endometrial stromal cell (HESC; n=4) cultures was confirmed by qPCR, with significantly decreased FKBP5 mRNA levels seen in FKBP5 siRNA-transfected cells (FkSi) vs control siRNA-transfected cells (Cont). DEX treatment increased FKBP5 mRNA levels in TM and HESC cell lines. This DEX-induced increase in FKBP5 levels was less pronounced in FKBP5-silenced cells (FkSi+DEX) for both cell lines. (FIG. 5B) DEX treatment increased HSD11B1 mRNA levels in control siRNA-transfected normal myometrial cell (TM; n=4), HESC (n=4), and leiomyoma (TLM; n=4) cells (DEX) vs vehicle treatment (Cont). This increase was blunted following FKBP5 knockdown (FkSi+DEX vs FkSi;-fold change relative to Cont). Bars represent mean ±standard error of the mean. *P<0.05.

FIG. 6A and FIG. 6B show that FKBP51 and dexamethasone induce a switch in leiomyoma cells from a smooth muscle to myofibroblast phenotype. (FIG. 6A) RNA-sequencing analysis of cultured leiomyoma cells (n=4) shows lower expression levels of extracellular matrix genes including LAMA2, LAMB1, and FN1, and higher expression levels of smooth muscle expressed genes CNN1, MYH9, MYH10, and ACTA2 following dexamethasone (DEX) treatment in FKBP5 siRNA (FKsi) vs control siRNA (Csi) transfected cells. (FIG. 6B) Confirmation of significantly lower expression levels of LAMA2 and FN1, and higher expression levels of CNN1 in FKsi-Dex vs Csi-Dex by qPCR analysis. Bars represent mean±standard error of the mean; n=4. RNA sequencing raw data available in the NCBI GEO repository, accession number GSE292403.

FIG. 7 shows a schematic illustration of the potential mechanism by which enhanced FKBP51-GR signaling contributes to the pathogenesis of leiomyoma. Elevated levels of FKBP51 augment glucocorticoid-induced GR-mediated upregulation of HSD11β1, which converts inactive cortisone to active cortisol, leading to sustained intracellular glucocorticoid signaling in leiomyoma cells. This FKBP51-GR-HSD11β1 relationship generates an intracellular pathological circle, leading to increased expression of extracellular matrix (ECM) genes and decreased expression scores of genes expressed by smooth muscle cells, supporting a switch from a smooth muscle to myofibroblast phenotype, thereby contributing to the pathogenesis of leiomyoma.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular examples only and is not intended to be limiting.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “about” and “approximately” are defined as being “close to” as understood by one of ordinary skill in the art. In one non-limiting example, the terms are defined to be within 10%. In another non-limiting example, the terms are defined to be within 5%. In still another non-limiting example, the terms are defined to be within 1%.

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genitourinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single-or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPSTM technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D-or L-amino acids or a mixture of D-and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01.

“Substitution” refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

In some examples, numbers expressing quantities of ingredients, properties such as molecular weight, reaction conditions, and so forth, used to describe and claim certain examples of the present disclosure are to be understood as being modified in some instances by the term “about.” In some examples, the term “about” is used to indicate that a value includes the standard deviation of the mean for the device or method being employed to determine the value. In some examples, the numerical parameters set forth in the written description and attached claims are approximations that can vary depending upon the desired properties sought to be obtained by a particular example. In some examples, the numerical parameters should be construed in light of the number of reported significant digits and by applying ordinary rounding techniques. Notwithstanding that the numerical ranges and parameters setting forth the broad scope of some examples of the present disclosure are approximations, the numerical values set forth in the specific examples are reported as precisely as practicable. The numerical values presented in some examples of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

The “tumor stroma” as used herein, is a critical component of the tumor microenvironment. It has crucial roles in supporting tumor initiation, progression, and metastasis by producing various growth factors, chemokines, and cytokines. Moreover, the dense collagen matrix of the tumor stroma is rich in cancer associated fibroblasts, which can contract and tighten the collagen network by secreting extracellular matrix associated with molecules and integrin dependent binding. Such a barrier not only physically prevents the access of therapeutic agents to reach tumor cells but also causes high interstitial fluid pressure to prevent the efficient discharge drugs and infiltration of immune cells in the tumor. Fibrotic tumor stroma is common in many stroma-rich human tumors, such as pancreatic, liver, colon, and triple negative breast cancers. There is experimental and clinical evidence that stroma-normalizing and therapeutic agents may synergize if administered in a specific sequence or combination. A tumor stroma with cells refers to the non-neoplastic supportive tissue associated with a tumor, comprising an extracellular matrix and one or more types of stromal cells. The “stromal cells” may include, but are not limited to, fibroblasts, immune cells (e.g., lymphocytes, macrophages), endothelial cells, and pericytes.

The current disclosure employs FKBP5 modulating agents, preferably oligonucleotides and similar species for use in modulating the function or effect of nucleic acid molecules encoding FK506-binding protein-51 (FKBP51). This is accomplished by providing oligonucleotides which specifically hybridize with one or more nucleic acid molecules of FKBP5 gene. As used herein, the terms “target nucleic acid” and “nucleic acid molecule of FKBP5 gene” have been used for convenience to encompass DNA of FKBP5 gene, RNA (including pre-mRNA and mRNA or portions thereof) transcribed from such DNA, and also cDNA derived from such RNA. The hybridization of a FKBP5 modulating agent of this disclosure with its target nucleic acid is generally referred to as “antisense”. Consequently, the preferred mechanism believed to be included in the practice of some preferred examples of the disclosure is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of FKBP5 gene. In the context of the current disclosure, “modulation” and “modulation of expression” mean either an increase (stimulation) or a decrease (inhibition) in the amount/levels/expression score/expression of a nucleic acid molecule encoding the gene, e.g., DNA or RNA. Inhibition is often the preferred form of modulation of expression and mRNA is often a preferred target nucleic acid.

In the context of this disclosure, “hybridization” means the pairing of complementary strands of oligomeric compositions. In the current disclosure, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compositions. For example, adenine and thymine are complementary nucleobases which pair through the formation of hydrogen bonds. Hybridization can occur under varying circumstances.

An antisense FKBP5 modulating agent composition is specifically hybridizable when binding of the composition to the target nucleic acid interferes with the normal function of the target nucleic acid to cause a loss of activity, and there is a sufficient degree of complementarity to avoid non-specific binding of the antisense composition to non-target nucleic acid sequences under conditions in which specific binding is desired, i.e., under physiological conditions in the case of in vivo assays or therapeutic treatment, and under conditions in which assays are performed in the case of in vitro assays.

In the current disclosure the phrase “stringent hybridization conditions” or “stringent conditions” refers to conditions under which a composition of the disclosure will hybridize to its target sequence, but to a minimal number of other sequences. Stringent conditions are sequence-dependent and will be different in different circumstances and in the context of this disclosure, “stringent conditions” under which oligomeric compositions hybridize to a target sequence are determined by the nature and composition of the oligomeric compositions and the assays in which they are being investigated.

“Complementary,” as used herein, refers to the capacity for precise pairing between two nucleobases of an oligomeric composition. For example, if a nucleobase at a certain position of an oligonucleotide (an oligomeric composition), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense composition need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compositions of the current disclosure comprise at least 70% sequence complementarity to a target region within the target nucleic acid, more preferably that they comprise 90% sequence complementarity and even more preferably comprise 95% sequence complementarity to the target region within the target nucleic acid sequence to which they are targeted. For example, an antisense composition in which 18 of 20 nucleobases of the antisense composition are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense composition which is 18 nucleobases in length having 4 (four) noncomplementary nucleobases which are flanked by two regions of complete complementarity with the target nucleic acid would have 77.8% overall complementarity with the target nucleic acid and would thus fall within the scope of the current disclosure. Percent complementarity of an antisense composition with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Uterine fibroids are benign monoclonal tumors originating from smooth muscle cells within the uterus. The exact initiating factor of fibroid formation remains unclear. However, prevailing theories highlight the role of reproductive hormones, inherent abnormalities of the myometrium, and predisposing genetic variations that affect cell signaling pathways. These tumors display altered glucocorticoid receptor activity, extracellular matrix accumulation, and smooth muscle hyperplasia. FK506 binding protein 51 is a co chaperone protein encoded by the FKBP5 gene. FKBP51 is known to associate with the glucocorticoid receptor and to modulate receptor sensitivity and transcriptional output. Uterine fibroids have an excessive amount of extracellular matrix (ECM) that makes up a significant portion of their bulk. While fibroids are made of smooth muscle cells, the key feature driving their growth and rigidity is the overproduction and disorganized deposition of ECM, primarily collagens, which is greater than what is found in normal uterine tissue. The hallmark of a uterine leiomyoma is the excessive production and deposition of ECM, such as collagens, fibronectin, and proteoglycans. This buildup makes the tumors stiff and contributes to their size and symptoms like pain and bleeding. Moreover, the tumor originates from the excessive growth of smooth muscle cells. However, the hyperplastic state combined with the excessive ECM production leads to the formation of the fibroid. Excessive ECM surrounds the cells and interacts with them in a way that promotes further growth and ECM production, creating a cycle that drives the tumor-like development.

Additionally, uterine leiomyomata are hormonally sensitive tumors, and evidence has demonstrated that leiomyomas overexpress certain estrogen and progesterone receptors when compared to normal surrounding myometrium. Studies indicate that the ovarian steroids estradiol and progesterone promote the growth of leiomyomas, and myometrial cells with high expression of estrogen and progesterone receptors can stimulate the growth of adjacent uterine leiomyomata stem cells in a paracrine fashion. Additionally, the size of uterine leiomyomata typically declines during the postpartum period, with the use of hormone-suppressing gonadotropin-releasing hormone (GnRH) analogs, and after menopause, when levels of those hormones fall.

Uterine fibroids are composed of monoclonal smooth muscle and fibroblast cells that have transformed at the microscopic level under the influence of sex steroid hormones. These abnormal cells feature upregulated expression of progesterone receptors (predominantly the PR-A isoform) and estrogen receptors (primarily ER-a). Histologic samples of uterine leiomyomata demonstrate a significant extracellular matrix (ECM) composed of collagen, proteoglycan, and fibronectin adjacent to disordered myometrium cells. Cells within a uterine leiomyomata typically have a low mitotic index. A thin layer of areolar tissue and compressed muscle fibers characteristically encapsulates uterine leiomyomata.

“Benign leiomyomas” are significantly more common than malignant leiomyosarcomas, and the conditions are differentiated based on their mitotic index, degree of cytologic atypia, and the presence of tumor cell necrosis. Leiomyomata demonstrate mild cytologic atypia, no tumor cell necrosis, and the mitotic activity is usually <5 mitotic figures per 10 high-power fields (hpf) in the most mitotically active areas. On the other hand, leiomyosarcomas demonstrate at least 2 of the following features: a mitotic index of at least 10 mitotic figures per 10 hpf, moderate to severe cytologic atypia, or the presence of tumor cell necrosis, also known as coagulative necrosis.

As used herein, “Myometrial smooth muscle cells (MSMC)” are located in the myometrium between the endometrium and the serosa of the uterus. MSMC play role in the stretching of the uterus during pregnancy and are also responsible for uterine contractions during labor. To accommodate the growing embryo, the myometrium expands through smooth muscle cell hypertrophy and hyperplasia. Typically, the uterine myometrium consists of differentiated smooth muscle cells, but in certain pathological conditions, such as uterine leiomyomata, smooth muscle cells in the myometrium proliferate abnormally to form tumors. Studies indicate that platelet-derived growth factor (PDGF) may stimulate MSMC proliferation and consequently may play a role in the formation of leiomyomata.

As used herein, “Hydroxysteroid 11 beta dehydrogenase 1 (HSD11β1)” refers to an enzyme that converts inactive cortisone to active cortisol, thereby amplifying local glucocorticoid availability. Elevated HSD11B1 expression has been documented in many glucocorticoid responsive tissues and contributes to pathological local cortisol reactivation. The inventors have identified a regulatory circuit in which FKBP51, glucocorticoid receptor activation, and induction of HSD11B1 mutually reinforce one another in leiomyoma cells. Mutations in this HSD11B1 gene can cause cortisone reductase deficiency. Alternate splicing results in multiple transcript variants encoding the same protein. HSD11B1 gene and protein sequence can be found on NCBI with reference sequence: NM_005525.4.

“Laminin subunit alpha 2 (LAMA2)” gene contributes to extracellular matrix architecture. Altered LAMA2 expression reflects changes in matrix composition characteristic of leiomyoma pathology. Laminin, an extracellular protein, is a major component of the basement membrane. It is thought to mediate the attachment, migration, and organization of cells into tissues during embryonic development by interacting with other extracellular matrix components. It is composed of three subunits, alpha, beta, and gamma, which are bound to each other by disulfide bonds into a cross-shaped molecule. This gene encodes the alpha 2 chain, which constitutes one of the subunits of laminin 2 (merosin) and laminin 4 (s-merosin). Mutations in this gene have been identified as the cause of congenital merosin-deficient muscular dystrophy. Two transcript variants encoding different proteins have been found for this gene. LAMA2 gene and protein sequence can be found on NCBI with reference sequence: NM_000426.4.

“Calponin 1 (CNN1)” gene encodes calponin 1 protein, a smooth muscle regulatory protein. Increased CNN1 expression score indicate enhanced contractile or myofibroblastic phenotype formation. CNN1 gene and protein sequence can be found on NCBI with reference sequence: NM_001299.6.

“Dexamethasone (DEX)” is a synthetic glucocorticoid used herein to investigate glucocorticoid receptor signaling strength and transcriptional output. DEX increases FKBP5 expression, reduces the number of cells in S-phase, and inhibits the expression of the estrogen receptor along with genes regulating cell replication in immortalized human uterine leiomyoma cells. Moreover, mifepristone, which is a competitive antagonist at both PR and GR with more than 3 times greater binding affinity for GR than DEX, inhibits extracellular matrix (ECM) formation in uterine leiomyoma. Together, these findings, along with our previous results, suggested that elevated glucocorticoid and/or progesterone signaling contribute to leiomyoma pathogenesis by increasing FKBP51 expression and promoting ECM formation.

“RNA sequencing” refers to high throughput sequencing based transcriptome analysis used to quantify gene expression changes induced by FKBP5 silencing or glucocorticoid stimulation.

“Quantitative polymerase chain reaction” is a method to real time amplification of specific transcripts to validate expression changes of selected genes.

As used herein, “FK506-binding-protein-51 (FKBP51)” is a co-chaperone protein encoded by the FKBP5 gene, which contains glucocorticoid and progesterone response elements, though FKBP5 is predominantly induced by glucocorticoids. Glucocorticoid-induced FKBP51 binds to both the glucocorticoid receptor (GR) and the progesterone receptor (PR), inhibiting GR-and PR-mediated transcriptional activity by impeding ligand binding. FKBP51 gene sequence can be found on NCBI with reference sequence: NM_004117.4 (Homo sapiens FKBP prolyl isomerase 5 (FKBP5), transcript variant 1, mRNA). FKBP51 amino acid sequence can be found on NCBI with reference sequence: NP_004108.1 peptidyl-prolyl cis-trans isomerase FKBP5 isoform 1 [Homo sapiens].

As used herein, “FKBP5 siRNA” refers to a small interfering RNA designed to specifically reduce expression of the FKBP5 transcript, thereby lowering intracellular FKBP51 protein expression. Control or scramble siRNA refers to a non targeted siRNA sequence used to demonstrate specificity. The FKBP5-specific siRNA was ordered from Santa Cruz®.

As used herein, “glucocorticoid receptor (GR)” refers to a nuclear receptor encoded by gene NR3C1. GR binds to glucocorticoid hormones such as cortisol and then change gene expression in response. In the female reproductive system, especially the uterus, this pathway is a major regulator of implantation, pregnancy maintenance, and timing of labor. In the absence of glucocorticoid hormone, GR sits in the cytoplasm with chaperone proteins like heat shock proteins (HSPs) and co chaperones such as FKBP51 and FKBP52. When cortisol or a synthetic glucocorticoid such as dexamethasone (DEX) binds GR, the receptor changes shape, moves into the nucleus, binds specific DNA sequences called glucocorticoid response elements, and regulates transcription of hundreds of genes. This entire process is referred to as GR signaling. It integrates signals from the hypothalamic pituitary adrenal axis with local tissue responses and is tightly modulated by enzymes such as HSD11B1 and HSD11B2 that control local cortisol levels. GR is widely expressed in uterine cell types including endometrial epithelium, stromal cells, smooth muscle cells in the myometrium, macrophages, and other immune cells.

GR signaling is important for functions such as implantation and normal endometrium maintenance. Local regeneration of cortisol by HSD11B1 in the endometrium provides ligand for GR and helps regulate genes that maintain a receptive endometrial environment and support early embryo survival. Additionally, human endometrial stromal cells upregulate HSD11B1 during decidualization, increasing local cortisol and GR activation. This GR signaling influences differentiation, cytokine production, and matrix remodeling in decidual cells. So, GR signaling contributes directly to the transition of stromal cells into specialized decidual cells that anchor and nourish the embryo. Glucocorticoid receptor functions via both genomic (predominant) and non-genomic pathways. The genomic pathway is mediated by GRs. In the absence of glucocorticoids, GRs exist principally anchored in the cytoplasm as part of a large complex of proteins, including chaperones (Hsp90, Hsp70, and p23) and immunophilins (FKBP51 and FKBP52), which help maintain GRs in a conformation that allows them to bind to ligands with high affinity [46]. Once glucocorticoids bind to GRs, receptor conformational changes lead to the dissociation of the protein complex and the exposure of 2 nuclear localization signals. The ligand-bound GR is then quickly transported into the nucleus through nuclear pores. Inside the nucleus, the GR binds directly to glucocorticoid response elements (GREs) as a dimer and activates the expression of target genes. This binding leads to further conformational changes in the GR, allowing it to recruit coregulators and chromatin-remodeling complexes that influence the activity of RNA polymerase II and activate gene transcription and repression. Binding to negative glucocorticoid-responsive elements (nGREs) can recruit corepressors (NCoR1 and SMRT) and histone deacetylases (HDACs) to repress the activity of target genes. In addition to its direct interaction with GREs, the GR can also interact with members of the signal transducer and activator of transcription (STAT) family to enhance transcription of specific target genes. The anti-inflammatory effects of glucocorticoids are mainly mediated by a negative regulatory mechanism called trans-repression. In this, the ligand-bound GR is recruited to genes by directly interacting with DNA-bound transcription factors, particularly NF-κB and activator protein-1 (AP-1). There are a number of proteins within the cell that can interact with the GR and potentiate trans-repression. For instance, O-linked-N-acetylglucosamine transferase (OGT), a placental stress biomarker, can interact with GRs and impact NF-κB-mediated transcription. The GR directly binds to the p65 subunit of NF-κB and the Jun subunit of AP-1, inhibiting their transcriptional activation. For some genes, GRs function in a composite manner, binding directly to a GRE and physically associating with AP1 or NF-κB bound to a neighboring site on the DNA, while for others, GRs bind to transcription factors without interacting directly with DNA.

The non-genomic effects of glucocorticoids, which occur without requiring gene transcription, are rapid and mediated through physiochemical interactions of cytosolic or membrane-bound GRs. These effects happen within seconds to minutes of activation and involve the activity of kinases such as phosphoinositide 3-kinase, AKT, Src kinase, mitogen-activated protein kinases (MAPKs), or intracellular Ca2+ signalling. Glucocorticoids can non-genomically interact with membrane lipids and proteins, cytoplasmic proteins, including various kinases, transcription factors, and glucocorticoid transporters, to influence cell function. High doses of glucocorticoids can directly interact with membrane lipids to alter membrane fluidity and affect ATP use and ion cyclicity. Glucocorticoids can also enhance Ca2+ signalling by binding to voltage-dependent calcium channels in brain synapses. For example, in cultured rat hippocampal cells, stress-induced elevated levels of corticosterone rapidly prolonged NMDA receptor-mediated Ca2+ signalling via the activation of a protein kinase C-dependent process by membrane GRs. Similarly, glucocorticoids have been found to non-genomically impact neurotransmission by affecting GABA, glutamate, serotonin, acetylcholine, and vasopressin receptors. Furthermore, glucocorticoids can engage with a variety of cytoplasmic proteins, including members of the MAPK family (extracellular signal-regulated protein kinases (ERK), c-Jun NH2-terminal protein kinase (JNK), and MAPK p38), phospholipases, and protein kinases, both genomically and non-genomically. MAPKs can regulate processes such as cell proliferation, development, differentiation, and apoptosis. Some examples of these interactions in relation to reproductive organs will be discussed in the forthcoming sections. Glucocorticoid administration to mouse thymocytes induced apoptosis via activation of phospholipase C, non-genomically, in vitro. Its administration to A549 cells in vitro prevented cell growth due to inhibition of prostaglandin E2 via a reduction in the activity of phospholipase A. Glucocorticoids can also secondarily activate MAPKs, Ca2+, and nitric oxide (NO) signaling and impact ion transport by activating protein kinases A, B, and C. Glucocorticoids can bind the extracellular site of membrane-bound GRs, which can then interact with and activate the stimulatory G protein, Gαs, to rapidly increase the levels of 3′,5′-cyclic adenosine monophosphate (cAMP), a common secondary messenger involved in the regulation of various biological processes and the actions of non-steroidal hormones. Lastly, when glucocorticoids bind to GRs, components of the multi-protein anchoring complex are released, which participate in secondary signaling pathways. The accessory proteins that have been reported to mediate these non-genomic effects of glucocorticoids include HSP-70, HSP-90, and MAPKs such as Src. Moreover, the ligand-bound GR can also interact with other proteins, including ZAP-90, leading to further downstream signaling.

As used herein, GR in complex with FKBP51, upregulates HSD11B1. HSD11B1 regenerates more active cortisol inside leiomyoma cells. Cortisol further activates GR and promotes additional FKBP51 expression. FKBP51-GR-HSD11β1 positive feedback loop that sustains and amplifies glucocorticoid signaling in leiomyomatous tissue. Enhanced GR signaling alters transcription of extracellular matrix and smooth muscle genes and promotes a switch from a typical smooth muscle phenotype toward a myofibroblast like phenotype, which is a hallmark of leiomyoma cells.

Decidualization is a biological transformation process that closely resembles a mesenchymal-epithelial transition (MET), occurring independently of the presence of an implanting blastocyst. Decidualizing cells differentiate from elongated fibroblast-like endometrial stromal cells (ESCs) into a more rounded and highly specialized secretory epithelioid cell type, termed decidual cells. Decidualizing ESCs undergo further morphological and biochemical alterations, including an expansion of the rough endoplasmic reticulum and Golgi complex, accumulation of glycogen and lipid droplets in the cytoplasm, enhanced expression of certain extracellular matrix proteins (laminin, type IV collagen, fibronectin, heparin sulfate), and an increase in the production of secretory proteins including prolactin (PRL) and insulin-like growth factor binding protein-1 (IGFBP-1), two established markers of decidualization.

“Endometrial disorders” as used herein, comprise Asherman's syndrome (AS), intrauterine scarring, fibrotic adhesions, menstrual disorders, pregnancy loss, infertility, leiomyomas, stromal endometriosis, endometrial stromal tumors. Endometrial stromal tumors are bundles of excessive cell growth originating within the uterine lining inside the uterus. Stromal endometriosis develops outside of the uterus and is defined as endometrial-like lesions on pelvic or abdominal structures.

“siRNA” plays a crucial role in gene silencing by following a multi-step process called RNA interference (RNAi). siRNA is created by cutting long double-stranded RNA molecules into shorter pieces that are then integrated into an RNA-induced silencing complex (RISC). Then, siRNA helps identify and bind to a matching messenger mRNA molecule, which is a copy of the gene that needs to be silenced. Once bound to the mRNA, the RISC, guided by siRNA, cuts the mRNA, thereby destroying it. With the mRNA degraded, it can no longer be used to make protein, leading to the silencing of the gene activity from which the mRNA was transcribed.

“Synthetic short interfering RNAs” could trigger gene silencing in mammalian cells, 1 there has been great interest in developing RNA interference (RNAi)-based technologies for both genetic study and therapeutic applications. siRNA molecules must be delivered intracellularly in order to trigger RNAi.

As used herein, “leiomyoma” refers to benign smooth muscle tumors derived from myometrial tissue. Leiomyoma exhibits extracellular matrix (ECM) accumulation, altered contractile protein expression, and aberrant glucocorticoid responsiveness. Leiomyomas typically contain increased expression of growth regulatory proteins, contractile markers, and metabolic enzymes relative to non-tumorous uterine tissue. Owing to their distinct molecular and physiological characteristics, leiomyomas serve as a representative model for assessing pathological smooth muscle behavior and aberrant glucocorticoid receptor signaling.

In certain examples, the term “uterine tissue sample” refers to any biological material obtained from the uterus and may include smooth muscle cells, stromal cells, extracellular matrix components, or mixed cell populations. Such samples can be collected from subjects with or without uterine pathology and may be processed as fresh tissue, cryopreserved tissue, cultured primary cells, or isolated nucleic acid and protein fractions.

“Normal myometrial tissue” as used herein, refers to the non-tumorous smooth muscle layer of the uterus that maintains characteristic cellular morphology, organized muscle fiber alignment, and balanced hormone responsive signaling. This tissue displays baseline transcriptional profiles associated with regulated proliferation, matrix turnover, and glucocorticoid receptor activity. Normal myometrium obtained from subjects without uterine abnormalities or from regions of the uterus distant from any lesion serves as a physiological reference for evaluating gene expression changes, protein levels/expression, or signaling perturbations.

“Paired myometrial tissue”, as used herein refers to non-tumorous uterine smooth muscle harvested from the same subject from whom a diseased sample, such as a leiomyoma, is collected. Because paired myometrium is exposed to identical hormonal and systemic factors as the corresponding diseased tissue, it provides an internal control that enables direct comparative assessment of transcriptional, biochemical, or functional differences attributable to localized pathological processes.

As used herein, “leiomyoma tissue” refers to benign smooth muscle tumors that originate within the myometrium. These tissues exhibit increased extracellular matrix accumulation, abnormal smooth muscle cell differentiation, elevated stress response protein expression, and altered steroid hormone responsiveness. Leiomyomas display distinct transcriptional and proteomic signatures compared to normal or paired myometrium, thereby providing a representative diseased uterine tissue model for assessing molecular mechanisms, gene regulatory networks, and therapeutic modulation strategies.

The ECM of fibroids consists of fibroblasts, often termed myofibroblasts, and reportedly producing a predominance of collagen types I and III. The “fibrous/collagenous” component that exists in these tumors, lends to the use of the colloquial derived terminology “fibroid”. The ECM may provide a reservoir for growth factors, cytokines, chemokines, angiogenic and inflammatory response mediators, and proteases produced by tumor cells, that are known to regulate events such as cell growth and differentiation, and ECM turnover, which are critical to leiomyoma growth and regression

In certain examples, human endometrial stromal cells (HESC) or myometrial cells isolated from subjects without leiomyoma may serve as additional control populations. These samples represent non-tumorous reference tissues for determining whether observed molecular alterations are specific to leiomyoma pathophysiology or reflect broader uterine biology. As used herein, HESCs display normal karyotype, respond to hormone stimulation, and retain the morphological pattern and biochemical endpoints of decidualization after treatment of estradiol and medroxyprogesterone, making this cell line a valuable control in various embodiments described herein.

Collectively, the use of normal myometrium, paired myometrium, leiomyoma tissues, and non-tumorous stromal or smooth muscle cells provides a structured sample framework for differentiating physiological uterine signaling from disease driven alterations. This framework enables assessment of gene expression changes, stress response pathways, and glucocorticoid regulatory mechanisms that contribute to uterine tumor development and progression.

In some examples, disclosed herein the primary leiomyoma tissues and leiomyoma adjacent myometrium display significantly elevated FKBP51 expression relative to normal myometrium. These tissues also exhibit increased HSD11B1 expression and enhanced sensitivity to glucocorticoid stimulation. The inventors investigated the molecular basis for this phenotype using RNA sequencing and quantitative polymerase chain reaction validation.

Leiomyoma cell cultures were transfected with either scramble siRNA or FKBP5 siRNA to reduce FKBP51 expression. After transfection, cultures were exposed to vehicle or dexamethasone. Differential transcriptional analysis revealed that HSD11B1 is strongly induced by dexamethasone in leiomyoma cells but that this induction is significantly reduced when FKBP51 is silenced. This indicates that FKBP51 does not simply inhibit glucocorticoid receptor function but can enhance glucocorticoid receptor mediated transcription of specific targets.

In addition, dexamethasone altered the expression of extracellular matrix and contractile genes in a FKBP51 dependent manner. Dexamethasone reduced LAMA2 expression and increased CNN1 expression more prominently in FKBP5 silenced cells compared to control cells. This indicates that FKBP51 coordinates glucocorticoid driven remodeling of matrix and cytoskeletal components.

These findings collectively support a mechanism in which FKBP51 elevates glucocorticoid receptor responsiveness to pathologically increase HSD11B1 expression. Increased HSD11B1 enhances intracellular cortisol regeneration, promoting persistent glucocorticoid receptor stimulation and transcriptional reprogramming toward increased extracellular matrix deposition and smooth muscle contractility. This represents a pathological FKBP51-GR-HSD11β1 circuit that contributes to leiomyoma growth and rigidity.

### Compositions

According to the current disclosure, FKBP5 modulating agents include antisense oligomeric compositions, antisense oligonucleotides, ribozymes, external guide sequence (EGS) oligonucleotides, alternate splicers, primers, probes, and other oligomeric compositions which hybridize to at least a portion of the target nucleic acid. As such, these compositions may be introduced in the form of single-stranded, double-stranded, circular or hairpin oligomeric compositions and may contain structural elements such as internal or terminal bulges or loops. Once introduced to a system, the compositions of the disclosure may elicit the action of one or more enzymes or structural proteins to effect modification of the target nucleic acid. One non-limiting example of such an enzyme is RNAse H, a cellular endonuclease which cleaves the RNA strand of an RNA: DNA duplex. It is known in the art that single-stranded antisense compositions which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense composition is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA expression (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

In the context of this disclosure, the term “oligomeric composition” refers to a polymer or oligomer comprising a plurality of monomeric units. In the context of this disclosure, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compositions of this disclosure, the current disclosure comprehends other families of compositions as well, including but not limited to oligonucleotide analogs and mimetics such as those described herein.

The compositions in accordance with this disclosure preferably comprise from about 8to about 80 nucleobases (i.e. from about 8 to about 80 linked nucleosides). One of ordinary skill in the art will appreciate that the disclosure embodies compositions of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred example, the compositions of the disclosure are 12 to 50 nucleobases in length. One having ordinary skill in the art will appreciate that this embodies compositions of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred example, the compositions of the disclosure are 15 to 30 nucleobases in length. One having ordinary skill in the art will appreciate that this embodies compositions of 15, 16, 17, 18, 19, 20, 21, 22, 23, 24, 25, 26, 27, 28, 29, or 30 nucleobases in length.

Particularly preferred compositions are oligonucleotides from about 12 to about 50 nucleobases, even more preferably those comprising from about 15 to about 30 nucleobases.

Antisense compositions 8-80 nucleobases in length comprising a stretch of at least eight (8) consecutive nucleobases selected from within the illustrative antisense compositions are considered to be suitable antisense compositions as well.

Exemplary preferred antisense compositions include oligonucleotide sequences that comprise at least the 8 consecutive nucleobases from the 5′-terminus of one of the illustrative preferred antisense compositions (the remaining nucleobases being a consecutive stretch of the same oligonucleotide beginning immediately upstream of the 5′-terminus of the antisense composition which is specifically hybridizable to the target nucleic acid and continuing until the oligonucleotide contains about 8 to about 80 nucleobases). Similarly preferred antisense compositions are represented by oligonucleotide sequences that comprise at least the 8 consecutive nucleobases from the 3′-terminus of one of the illustrative preferred antisense compositions (the remaining nucleobases being a consecutive stretch of the same oligonucleotide beginning immediately downstream of the 3′-terminus of the antisense composition which is specifically hybridizable to the target nucleic acid and continuing until the oligonucleotide contains about 8 to about 80 nucleobases). One having skill in the art armed with the preferred antisense compositions illustrated herein will be able, without undue experimentation, to identify further preferred antisense compositions.

“Targeting” an antisense composition to a particular nucleic acid molecule, in the context of this disclosure, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In the current disclosure, the target nucleic acid comprises FKBP5 gene.

The targeting process usually also includes determination of at least one target region, segment, or site within the target nucleic acid for the antisense interaction to occur such that the desired effect, e.g., modulation of expression, will result. Within the context of the current disclosure, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the current disclosure, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the disclosure, “start codon” and “translation initiation codon” refer to the codon or codons that are used in vivo to initiate translation of an mRNA transcribed from a gene of FKBP5 gene, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compositions of the current disclosure.

The open reading frame (ORF) or “coding region,” which is known in the art to refer to the region between the translation initiation codon and the translation termination codon, is also a region which may be targeted effectively. Within the context of the current disclosure, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compositions targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the disclosure, the types of variants described herein are also preferred target nucleic acids.

The locations on the target nucleic acid to which the preferred antisense compositions hybridize are hereinbelow referred to as “preferred target segments.” As used herein the term “preferred target segment” is defined as at least an 8-nucleobase portion of a target region to which an active antisense composition is targeted. While not wishing to be bound by theory, it is presently believed that these target segments represent portions of the target nucleic acid which are accessible for hybridization.

While the specific sequences of certain preferred target segments are set forth herein, one of skill in the art will recognize that these serve to illustrate and describe particular examples within the scope of the current disclosure. Additional preferred target segments may be identified by one having ordinary skill.

Target segments 8-80 nucleobases in length comprising a stretch of at least eight (8) consecutive nucleobases selected from within the illustrative preferred target segments are considered to be suitable for targeting as well.

Target segments can include DNA or RNA sequences that comprise at least the 8 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 8 to about 80 nucleobases). Similarly preferred target segments are represented by DNA or RNA sequences that comprise at least the 8 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 8 to about 80 nucleobases). One having skill in the art armed with the preferred target segments illustrated herein will be able, without undue experimentation, to identify further preferred target segments.

Once one or more target regions, segments or sites have been identified, antisense compositions are chosen which are sufficiently complementary to the target, i.e., hybridize sufficiently well and with sufficient specificity, to give the desired effect.

In one example, disclosed herein is a pharmaceutical composition, comprising: a FKBP5 modulating agent; and a therapeutically acceptable carrier.

In some examples, the FKBP5 modulating agent comprises an FKBP5 small interfering RNA (siRNA).

In some examples, the FKBP5 siRNA comprises 8-30 nucleotides targeting a nucleic acid molecule encoding FKBP5 gene.

In some examples, the FKBP5 modulating agent comprises a small molecule inhibitor of FKBP51 protein activity.

In some examples, the FKBP5 modulating agent comprises an inhibitor of HSD11B1.

In some examples, the therapeutically acceptable carrier comprises polymer micelle, liposome, emulsion, microsphere, or nanosphere.

In some examples, the FKBP5 modulating agent is an antisense oligonucleotide, wherein the antisense oligonucleotide is a DNA oligonucleotide, an RNA oligonucleotide, or a chimeric oligonucleotide.

In some examples, the antisense oligonucleotide hybridizes with an FKBP5 RNA to form an oligonucleotide-RNA duplex.

In some examples, the FKBP5 modulating agent shares at least 70% complementarity with a nucleic acid molecule encoding FKBP5 gene. In some examples, the FKBP5 modulating agent shares at least 80% complementarity with a nucleic acid molecule encoding FKBP5 gene. In some examples, the FKBP5 modulating agent shares at least 90% complementarity with a nucleic acid molecule encoding FKBP5 gene. In some examples, the FKBP5 modulating agent shares at least 95% complementarity with a nucleic acid molecule encoding FKBP5 gene.

In an example, disclosed herein is a composition comprising:
a. a nucleic acid molecule that inhibits HSD11B1 expression; and
b. a delivery vehicle selected from lipid nanoparticles, viral vectors, non viral polymeric carriers, or extracellular vesicles for reducing HSD11B1 expression in uterine fibroid tissue.

In some examples, the nucleic acid molecule comprises a sequence that is complementary to an HSD11B1 transcript.

In some examples, the delivery vehicle targets smooth muscle cells of the uterus.

In an example, disclosed herein is a composition comprising: an HSD11B1 inhibitor; and a pharmaceutically acceptable carrier, for reducing fibroid growth or activity in a subject.

### Modifications to the Compositions

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compositions are generally preferred. In addition, linear compositions may have internal nucleobase complementarity and may therefore-fold in a manner as to produce a fully or partially double-stranded FKBP5 modulating agent. Within oligonucleotides, the phosphate groups are commonly referred to as forming the internucleoside backbone of the oligonucleotide. The normal linkage or backbone of RNA and DNA is a 3′ to 5′ phosphodiester linkage.

Modified Internucleoside Linkages (backbones)

Specific examples of preferred antisense compositions useful in this disclosure include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

### Modified Sugar and Internucleoside Linkages-mimetics

In other preferred oligonucleotide mimetics, both the sugar and the internucleoside linkage (i.e. the backbone), of the nucleotide units are replaced with novel groups. The nucleobase units are maintained for hybridization with an appropriate target nucleic acid. One such FKBP5 modulating agent, an oligonucleotide mimetic that has been shown to have excellent hybridization properties, is referred to as a peptide nucleic acid (PNA). In PNA compositions, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compositions include, but are not limited to, U.S. Pat. Nos. 5,539,082; 5,714,331; and 5,719,262, each of which is herein incorporated by reference. Further teaching of PNA compositions can be found in Nielsen et al., Science, 1991, 254, 1497-1500.

Preferred examples of the disclosure are oligonucleotides with phosphorothioate backbones and oligonucleosides with heteroatom backbones, and in particular —CH₂—NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

### Modified Sugars

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S—or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH2—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

### Natural and Modified Nucleobases

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b] benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the compositions of the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

### Conjugates

Another modification of the oligonucleotides of the disclosure involves chemically linking to the oligonucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compositions of the current disclosure. Representative conjugate groups are disclosed in International Patent Application PCT/US 92/09196, filed Oct. 23, 1992, and U.S. Pat. No. 6,287,860, the entire disclosure of which are incorporated herein by reference. Conjugate moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligonucleotides of the disclosure may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

### Chimeric Compositions

It is not necessary for all positions in a given composition to be uniformly modified, and in fact more than one of the aforementioned modifications may be incorporated in a single composition or even at a single nucleoside within an oligonucleotide.

In some examples, disclosed herein are FKBP5 modulating agents include but not limited to chimeric compositions. “Chimeric” antisense compositions or “chimeras,” in the context of this disclosure, are antisense compositions, particularly oligonucleotides, which contain two or more chemically distinct regions, each made up of at least one monomer unit, i.e., a nucleotide in the case of an oligonucleotide composition. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA: RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compositions of the disclosure may be formed as composite structures of two or more oligonucleotides, modified oligonucleotides, oligonucleosides and/or oligonucleotide mimetics as described above. Such compositions have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

### Methods of Treatment

Currently only limited therapeutic options are available to treat uterine disorders. Some of the therapies available comprise clinical management of uterine fibroids aimed at controlling symptoms (bleeding, pain, bulk) and reducing fibroid size.

The non-hormonal therapy options are limited and mainly work on the symptoms caused by uterine fibroids rather than their cause. The non-hormonal treatment options include nonsteroidal anti-inflammatory drugs (NSAIDs), such as ibuprofen, to reduce menstrual pain and bleeding, or tranexamic acid, an antifibrinolytic used during to reduce heavy menstrual bleeding.

Several hormonal options are available, but they mainly work by suppressing ovarian steroid production or modifying progesterone and estrogen signaling. The sex hormone options include combined oral contraceptives and progestin only therapies used to control bleeding and effect fibroid volume; levonorgestrel releasing intrauterine system (LNG IUS) which provides high local progestin levels in the uterus, reduces heavy menstrual bleeding, and can be used in women with fibroids that do not markedly distort the cavity; gonadotropin releasing hormone (GnRH) agonists like leuprolide to induce a reversible hypoestrogenic state, and shrink fibroids; oral GnRH antagonists such as elagolix and relugolix, combined with low dose estrogen and progestin to reduce heavy menstrual bleeding and fibroid size; selective progesterone receptor modulators (SPRMs) like ulipristal acetate to reduce bleeding and shrinking fibroids; aromatase inhibitors and other endocrine agents.

The other options available to treat leiomyomas and other uterine disorders involve surgical options including but not limited to myomectomy, hysterectomy, uterine artery embolization, radiofrequency ablation or MR guided focused ultrasound

At present, there is no approved drug specifically designed to inhibit GR, FKBP51, or HSD11B1 in leiomyomas. None of the approved therapies are designed specifically to break the FKBP51-GR-HSD11β1 loop.

As used herein, the methods for treating a disease or condition relates to the therapeutic use of FKBP5 modulating agents. In this respect, the FKBP5 modulating agents may be administered to a subject having an existing disease or condition in order to lessen, reduce or improve at least one symptom associated with the disease or condition and/or to slow down, reduce or block the progression of the disease.

Suitably, treating and/or preventing an autoimmune or inflammatory disease may refer to administering an effective amount of the FKBP5 modulating agent (e.g., FKBP5 siRNA) such that the amount of existing medication (e.g. exogenous insulin) that a subject with said disease requires is reduced, or may enable the discontinuation of the subject's existing medication.

Preventing a disease or condition relates to the prophylactic use of the FKBP5 modulating agents herein. In this respect, the cells may be administered to a subject who has not yet contracted or developed the disease or condition and/or who is not showing any symptoms of the disease or condition to prevent the disease or condition or to reduce or prevent development of at least one symptom associated with the disease or condition. The subject may have a predisposition for, or be thought to be at risk of developing, the disease or condition (e.g. uterine leiomyoma).

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to affect a measurable improvement of a particular disease, disorder, or condition. A therapeutically effective amount herein may vary according to factors such as the disease state, age, sex, and weight of the patient, and the ability of the chimeric receptors to elicit a desired response in the individual. A therapeutically effective amount is also one in which any toxic or detrimental effects of the FKBP5 modulating agents including siRNA, miRNA, gene therapy, viral vectors, genetically modified cell, cell population or pharmaceutic compositions are outweighed by the therapeutically beneficial effects.

The terms “subject”, “patient” and “individual” are used interchangeably herein and refer to a mammal, optionally a human. In particular, the terms subject, patient and individual refer to a human having a disease or disorder as defined herein in need of treatment.

In one example, disclosed herein is a method of treating a uterine disorder in a subject, comprising:
determining an expression score of one or more biomarkers in a biological sample obtained from a subject relative to a reference control,
   wherein the one or more biomarkers comprise one or more RNAs, or an expression product thereof, of the genes selected from the group consisting of TSC22D3, ADH1B, IL1R1, HSD11B1, GREM1, IGFBP5, AMIGO2, and FKBP5;
determining a cumulative expression score from the one or more expression scores of the one or more biomarkers; and
administering to the subject a therapeutically effective amount of an FKBP5-modulating agent if the cumulative expression score is lower than a threshold.

RNA seq differential expression in the current disclosure is determined using statistical modeling of normalized counts rather than mean or median values, with DEGs defined by absolute log 2 fold change greater than 1. IPA pathway predictions apply a Z score threshold where values greater than 2 indicate activation and values less than minus 2 indicate inhibition. qPCR validation uses the mean of technical replicates to calculate ΔCt, ΔΔCt, and fold change, in which a value greater than 1 denotes upregulation and a value less than 1 denotes downregulation. The threshold range therefore depends on the analytical step: +1 or −1 for log2 fold change in RNA seq, plus 2 or minus 2 for Z score interpretation, and fold change greater or less than 1 for qPCR based expression scoring.

Calculation of the gene expression levels or expression scores are determined using quantitative PCR, wherein the amplification cycle threshold (Ct) reflects the abundance of a target nucleic acid in a biological sample. Ct values typically range from 10 to 40 cycles, with lower Ct values indicating higher expression and higher Ct values indicating lower expression. Ct values below approximately 15 cycles correspond to highly abundant transcripts, Ct values between approximately 15 and 30 cycles represent moderate expression, and Ct values above approximately 35 cycles indicate low or near background expression. Ct values are unitless measures representing the number of amplification cycles required to reach a predefined fluorescence threshold.

In additional examples, gene expression levels or expression scores are determined using RNA sequencing, and differential expression is reported as a log2 fold change relative to a reference control. A log2 fold change of 1 corresponds to a two-fold increase in expression, a log2 fold change of minus 1 corresponds to a two-fold decrease, and values above or below these thresholds reflect proportionally larger increases or decreases. Log2 fold change values are unitless and provide a standardized measure of the magnitude and direction of expression differences across samples.

In some examples, Ct based fold changes and RNA sequencing based log2 fold changes are converted into an expression score for each biomarker. The expression score provides a normalized, unitless measurement of gene expression relative to the reference control. An expression score of 1 indicates no change, an expression score of 2 or greater indicates an increase of at least two-fold, and an expression score of 0.5 or lower indicates a decrease of at least two-fold relative to the reference control.

In certain examples, when a panel of two or more biomarkers is used, an expression score is calculated for each biomarker individually and the mean of the individual expression scores is determined to generate a panel cumulative expression score. Calculation of the mean is appropriate when the diagnostic or prognostic method relies on the collective behavior of the biomarker panel rather than on a single marker. The mean expression score typically ranges from approximately 0.1 to 10, depending on the degree of downregulation or upregulation of the constituent biomarkers. A mean expression score greater than or equal to 2 indicates coordinated induction of the biomarker panel, whereas a mean expression score of 0.5 or lower indicates coordinated suppression. Intermediate values between 0.5 and 2 indicate partial or absent activation of the biomarker panel.

In additional examples, the mean expression score is compared to a predetermined threshold to classify a subject, diagnose a disorder, or select a therapeutic intervention. The predetermined threshold may comprise a value of 2 or greater for induced biomarker panels, a value of 0.5 or lower for suppressed biomarker panels, or any value between 0.5 and 2 depending on the required sensitivity or specificity of the diagnostic algorithm.

In some examples, the expression score greater than 1 indicates upregulation relative to the reference control and an expression score less than 1 indicates downregulation relative to the reference control. In some examples, the quantifying is carried out by one or a combination of Polymerase Chain Reaction, Real Time-Polymerase Chain Reaction, Real Time Reverse Transcriptase-Polymerase Chain Reaction, Real-time quantitative RT-PCR, Northern blot analysis, in situ hybridization, or probe array.

In the current disclosure, several types of biological reference controls are used depending on the experiment. For qPCR analyses, gene expression is first normalized internally to the endogenous housekeeping controls ACTB and GAPDH, and all expression scores are calculated using the 2^{-ΔΔCT} method. For dexamethasone-response experiments in leiomyoma cultures, the biological reference control is the vehicle-treated, control siRNA-transfected leiomyoma cells, which serve as the calibrator from which all dexamethasone-induced-fold changes are derived. FKBP5 knockdown efficiency studies use scramble siRNA without dexamethasone as the reference control. For tissue comparisons measuring HSD11B1 expression in normal myometrium, paired myometrium, and leiomyoma, the reference control is normal myometrium from patients without leiomyoma, providing the physiological baseline for-fold-change calculations. In comparisons across different uterine cell types, including leiomyoma cells, normal myometrial cells, and human endometrial stromal cells, the vehicle-treated control siRNA condition for each respective cell type serves as the reference control for quantifying dexamethasone-induced HSD11B1 expression. For RNA-sequencing analyses, control siRNA plus vehicle (C) is the reference control for C-DEX vs C comparisons, FKBP5 siRNA plus vehicle (FK or Fksi) serves as the reference for FK-DEX vs FK comparisons, and the control siRNA plus dexamethasone condition is the reference when comparing FKBP5-silenced dexamethasone-treated cells to dexamethasone-treated controls. Expression scores throughout the study represent relative expression values generated by normalizing each biomarker to ACTB/GAPDH and calculating-fold-change against the relevant biological reference control described above. The thresholds observed in the study reflect typical glucocorticoid-responsive behavior, in which dexamethasone-induced genes exhibit several-fold increases under normal signaling conditions and dexamethasone-suppressed genes fall well below baseline, whereas FKBP5 knockdown consistently blunts these responses, reducing upregulation or downregulation by more than half. These experimentally defined-fold-change patterns provide the basis for establishing clinically useful thresholds in diagnostic or treatment-selection methods.

In some examples, the FKBP5 modulating agent comprises an FKBP5 small interfering RNA (siRNA).

In some examples, the FKBP5 modulating agent comprises a small molecule inhibitor of FKBP51 protein activity.

In some examples, the FKBP5 modulating agent comprises an inhibitor of HSD11B1.

In some examples, the biological sample comprises leiomyoma tissue, paired myometrial tissue, myometrial tissue, endometrial stromal cells, or any combination thereof.

In some examples, the uterine disorders comprise uterine leiomyoma, uterine fibroids, abnormal uterine bleeding, myometrial hyperplasia, progesterone resistant uterine disorder, estrogen responsive uterine disorder, endometriosis, pelvic pain, benign adnexal mass, adenomyosis, uterine cancer, polycystic ovary syndrome (PCOS) or cervical dysplasia.

Examples of uterine disorders include but are not limited to uterine fibroids, also known as leiomyomas, are benign (non-cancerous) growths of muscle and connective tissue in the uterus. They are among the most common uterine disorders, affecting up to 70-80% of women during their lifetime. Fibroids can range in size from very small to large masses, and they may not cause symptoms. However, when they do, symptoms can include heavy menstrual bleeding, pelvic pressure or pain, frequent urination, and difficulty emptying the bladder; Endometrial polyps are growths of tissue that form on the inner lining of the uterus (the endometrium). These growths are usually benign but can cause abnormal bleeding, including heavy periods or spotting between periods. While most polyps are not cancerous, some may be associated with an increased risk of endometrial cancer, especially in postmenopausal women; Adenomyosis occurs when the tissue that normally lines the uterus (the endometrium) grows into the muscular wall of the uterus. This condition can cause the uterus to enlarge and may result in heavy periods, painful periods, and pelvic pain. Adenomyosis is often confused with fibroids, but the two are distinct conditions. While the exact cause is unknown, it is more common in women who have had children or who are in their 40s or 50s; Uterine septum is a congenital condition in which the uterus is divided by a wall of tissue (septum). This can cause complications in pregnancy, including miscarriage or preterm birth. While many women with a uterine septum are asymptomatic, it is important to diagnose and manage this condition, especially for those experiencing fertility issues; or Uterine leiomyosarcoma is a malignant tumor that originates from the smooth muscle of the uterus. It can be challenging to distinguish from benign fibroids, so it's important to seek medical evaluation for any concerning symptoms. Symptoms may include abnormal bleeding, pelvic pain, and bloating.

In one example, disclosed herein is a method of treating a uterine disorder in a subject, comprising:
determining an expression score of a first group of one or more biomarkers, a second group of one or more biomarkers, a third group of one or more biomarkers, and a fourth group of one or more biomarkers in a biological sample obtained from a subject relative to a reference control,
wherein the first group of one or more biomarkers comprise one or more RNAs, or an expression product thereof, of the genes selected from the group consisting of CNN1, MYH9, MYH10, and ACTA2, the second group of one or more biomarkers comprise one or more RNAs, or an expression product thereof, of the genes selected from the group consisting of GREM1, IGFBP5, and AMIGO2, the third group of one or more biomarkers comprise one or more RNAs, or an expression product thereof, of the genes selected from the group consisting of LAMA2, LAMB1, and FN1, and the fourth group of one or more biomarkers comprise one or more RNAs, or an expression product thereof, of the genes selected from the group consisting of TSC22D3, ADH1B, IL1R1, and HSD11B1;
determining a first expression score by integrating the expression scores of the one or more biomarkers in the first group, a second expression score by integrating the expression score of the one or more biomarkers in the second group, a third expression score by integrating the expression score of the one or more biomarkers in the third group, and a fourth expression score by integrating the expression score of the one or more biomarkers in the fourth group; and
administering to the subject a therapeutically effective amount of an FKBP5 modulating agent if the first expression score is lower than a first threshold, the second expression score is lower than a second threshold, the third expression score is higher than a third threshold, and the fourth expression score is higher than a fourth threshold.

In some examples, wherein the therapeutically effective amount of the FKBP5 modulating agent is administered to the subject intravenously, intramuscularly, intraperitoneally, intradermally, or subcutaneously. In some examples, the therapeutically effective amount is administered as at least a single dose or multiple doses, wherein a single dose is administered at a concentration of at least 0.005 mg/kg/day to 30 mg/kg/day, or at least from 0.01 mg/kg/day to 10 mg/kg/day, or from 0.01 mg/kg/day to 2 mg/kg/day.

Some examples comprise administering to the subject a therapeutically effective amount of the FKBP5 modulating agent if the first expression score is lower than 0.5, the second expression score is lower than 0.5, the third expression score is higher than 2, and the fourth expression score is higher than 2.

In some examples, the biological sample from the subject has a higher expression of FKBP51 or HSD11β1 protein as compared to the reference control.

In some examples, the FKBP5 modulating agent comprises an FKBP5 small interfering RNA (siRNA). In some examples, the siRNA is administered in combination with at least one anti-angiogenic agent, an anti-tumor agent, an immunotherapeutic agent, or a combination thereof.

In some examples, the FKBP5 modulating agent comprises a small molecule inhibitor of FKBP51 protein activity. In some examples, the FKBP5 modulating agent comprises an oligonucleotide, an antisense oligonucleotide, a DNA oligonucleotide, an RNA oligonucleotide, an RNA oligonucleotide having at least a portion of said RNA oligonucleotide capable of hybridizing with RNA to form an oligonucleotide-RNA duplex, or a chimeric oligonucleotide. In some examples, the FKBP5 antisense oligonucleotide comprises at least one modified internucleoside linkage, sugar moiety, or nucleobase. In some examples, the FKBP5 antisense oligonucleotide comprises at least one 2′-O-methoxyethyl sugar moiety. In some examples, the FKBP5 antisense oligonucleotide comprises at least one phosphorothioate internucleoside linkage. In some examples, the FKBP5 antisense oligonucleotide comprises at least one 5-methylcytosine.

In some examples, the FKBP5 modulating agent comprises an inhibitor of HSD11B1.

In some examples, the biological sample comprises leiomyoma tissue, paired myometrial tissue, myometrial tissue, endometrial stromal cells, or any combination thereof.

In some examples, the uterine disorders comprise uterine leiomyoma, uterine fibroids, abnormal uterine bleeding, myometrial hyperplasia, progesterone resistant uterine disorder, estrogen responsive uterine disorder, endometriosis, pelvic pain, benign adnexal mass, adenomyosis, uterine cancer, polycystic ovary syndrome (PCOS) or cervical dysplasia.

In an example, disclosed herein is a method for detecting uterine fibroid tissue in a subject comprising:
measuring the expression score of HSD11B1 in a sample; and
identifying the sample as fibroid tissue when the HSD11B1 expression score is elevated relative to non fibroid uterine tissue.

In some examples, the HSD11B1 expression score is measured by PCR, RNA sequencing, immunoassay, or enzymatic activity assay.

In some examples, the identification is performed by comparing the measured expression score to a threshold reference value derived from healthy uterine tissue.

In an example, disclosed herein is a method for treating a uterine fibroid in a subject comprising:
a. administering to the subject an inhibitor of HSD11B1 expression or activity,
b. thereby reducing glucocorticoid activation within fibroid tissue.

In some examples, the inhibitor is a small molecule, antisense oligonucleotide, siRNA, shRNA, CRISPR reagent, monoclonal antibody, or peptide inhibitor.

In some examples, the administration of the inhibitor decreases cortisol levels or glucocorticoid receptor activation in the fibroid tissue.

In an example, disclosed herein is a method for modulating HSD11B1 expression in uterine fibroid cells in a subject comprising: delivering to the fibroid cells a gene modulation agent that reduces HSD11B1 expression; and decreasing glucocorticoid activation within the fibroid cells as a result of the reduced HSD11B1 expression.

In some examples, the gene modulation agent comprises an siRNA, shRNA, antisense oligonucleotide, microRNA mimic, microRNA inhibitor, CRISPR interference construct, or a transcriptional repressor.

In some examples, the agent specifically binds to an HSD11B1 messenger RNA sequence and induces degradation or translational repression of the sequence.

In some examples, the agent comprises a CRISPR system configured to silence HSD11B1 expression by blocking transcription initiation or elongation through a nuclease deficient Cas protein fused to a repressor domain.

In some examples, the decreased glucocorticoid activation results in reduced local cortisol levels in the fibroid cells.

In an example, disclosed herein is a method for treating a uterine fibroid in a subject comprising:
a. administering a composition comprising HSD11B1 inhibitor to the subject; and
b. reducing fibroid size or activity by lowering HSD11B1 mediated glucocorticoid signaling.

In an example, disclosed herein is a method for reducing glucocorticoid receptor activation in uterine fibroid cells comprising: delivering to the cells a CRISPR based construct that disrupts transcription of the HSD11B1 gene.

In some examples, the construct comprises a guide RNA targeting the HSD11B1 promoter region and a deactivated Cas protein fused to a KRAB or other transcriptional repressor domain.

### Screening and Target Validation

In a further example, the “preferred target segments” identified herein may be employed in a screen for additional compositions that modulate the expression of FKBP5 gene. “Modulators” are those compositions that decrease or increase the expression of a nucleic acid molecule of FKBP5 gene, and which comprise at least an 8-nucleobase portion which is complementary to a preferred target segment. The screening method comprises the steps of contacting a preferred target segment of a nucleic acid molecule of FKBP5 gene with one or more candidate modulators and selecting for one or more candidate modulators which decrease or increase the expression of a nucleic acid molecule of FKBP5 gene. Once it is shown that the candidate modulator or modulators are capable of modulating (e.g. either decreasing or increasing) the expression of a nucleic acid molecule of FKBP5 gene, the modulator may then be employed in further investigative studies of the function of FKBP5 gene, or for use as a research, diagnostic, or therapeutic agent in accordance with the current disclosure.

The preferred target segments of the current disclosure may be also be combined with their respective complementary antisense compositions of the current disclosure to form stabilized double-stranded (duplexed) oligonucleotides.

Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The compositions of the current disclosure can also be applied in the areas of drug discovery and target validation. The current disclosure comprehends the use of the compositions and preferred target segments identified herein in drug discovery efforts to elucidate relationships that exist between FKBP5 gene and a disease state, phenotype, or condition. These methods include detecting or modulating FKBP5 gene comprising contacting a sample, tissue, cell, or organism with the compositions of the current disclosure, measuring the nucleic acid or protein expression score of FKBP5 gene and/or a related phenotypic or chemical endpoint at some time after treatment, and optionally comparing the measured value to a non-treated sample or sample treated with a further composition of the disclosure. These methods can also be performed in parallel or in combination with other experiments to determine the function of unknown genes for the process of target validation or to determine the validity of a particular gene product as a target for treatment or prevention of a particular disease, condition, or phenotype.

In one example, disclosed herein is a method of inhibiting the expression of FKBP5 gene or HSD11B1 gene in cells or tissues comprising contacting said cells or tissues with an inhibitor or modulating agent comprising antisense oligonucleotides or a pharmaceutically effective composition to inhibit the expression of FKBP5 or HSD11B1.

In one example, disclosed herein is a method of screening for a FKBP5 modulating agent, wherein the method comprises: contacting a preferred target segment of a nucleic acid molecule encoding FKBP5 gene with one or more candidate modulators of FKBP5 gene, and identifying one or more modulators of FKBP5 gene expression which modulate the expression of FKBP5 gene.

In an example, disclosed herein is a method for stratifying subjects for fibroid treatment comprising:
a. measuring HSD11B1 expression in uterine tissue;
b. classifying the subject as high glucocorticoid signaling if the HSD11B1 expression exceeds a predetermined value; and
c. determining an HSD11B1 targeted therapy for the high signaling subject.

### Formulations

The compositions of the disclosure may also be admixed, encapsulated, conjugated or otherwise associated with other molecules, molecule structures or mixtures of compositions, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compositions of the disclosure encompass any pharmaceutically/therapeutically acceptable salts, esters, or salts of such esters, or any other composition which, upon administration to an animal, including a human, is capable of providing (directly or indirectly) the biologically active metabolite or residue thereof. Accordingly, for example, the disclosure is also drawn to prodrugs and pharmaceutically/therapeutically acceptable salts of the compositions of the disclosure, pharmaceutically/therapeutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the disclosure are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically/therapeutically acceptable salts” refers to physiologically and pharmaceutically/therapeutically acceptable salts of the compositions of the disclosure: i.e., salts that retain the desired biological activity of the parent composition and do not impart undesired toxicological effects thereto. For oligonucleotides, preferred examples of pharmaceutically/therapeutically acceptable salts and their uses are further described in U.S. Pat. No. 6,287,860, which is incorporated herein in its entirety.

The current disclosure also includes therapeutic/pharmaceutical compositions and formulations which include the antisense compositions of the disclosure. The pharmaceutical compositions of the current disclosure may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical/therapeutically acceptable carriers, aqueous, powder or oily bases, thickeners and the like may be necessary or desirable. Coated condoms, gloves and the like may also be useful.

The pharmaceutical formulations of the current disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the current disclosure may be formulated into any of many possible dosage forms such as, but not limited to, tablets, capsules, gel capsules, liquid syrups, soft gels, suppositories, and enemas. The compositions of the current disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the current disclosure include, but are not limited to, solutions, emulsions, foams and liposome-containing formulations. The pharmaceutical compositions and formulations of the current disclosure may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an example of the current disclosure. Emulsions and their uses are well known in the art and are further described in U.S. Pat. No. 6,287,860, which is incorporated herein in its entirety.

Formulations of the current disclosure include liposomal formulations. As used in the current disclosure, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the current disclosure may also include surfactants. The use of surfactants in drug products, formulations and in emulsions is well known in the art. Surfactants and their uses are further described in U.S. Pat. No. 6,287,860, which is incorporated herein in its entirety.

In one example, the current disclosure employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the disclosure are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the disclosure may be encapsulated within liposomes or may form complexes thereto, in particular to cationic liposomes. Alternatively, oligonucleotides may be complexed to lipids, in particular to cationic lipids. Preferred fatty acids and esters, pharmaceutically/therapeutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticles, nanoparticles, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the disclosure are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the disclosure may be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are described in detail in U.S. applications Ser. Nos. Ser. No. 09/108,673 (filed Jul. 1, 1998), Ser. No. 09/315,298 (filed May 20, 1999) and Ser. No. 10/071,822, filed Feb. 8, 2002, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compositions and other pharmaceutically/therapeutically acceptable carriers or excipients.

Certain examples of the disclosure provide pharmaceutical compositions containing one or more oligomeric compositions and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compositions of the disclosure, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the disclosure. Combinations of antisense compositions and other non-antisense drugs are also within the scope of this disclosure. Two or more combined compositions may be used together or sequentially.

In another related example, compositions of the disclosure may contain one or more antisense compositions, particularly oligonucleotides, targeted to a first nucleic acid and one or more additional antisense compositions targeted to a second nucleic acid target. Alternatively, compositions of the disclosure may contain two or more antisense compositions targeted to different regions of the same nucleic acid target. Numerous examples of antisense compositions are known in the art. Two or more combined compositions may be used together or sequentially.

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

Administration of the compositions of the invention may be carried out in any convenient manner, including by aerosol inhalation, injection, ingestion, infusion, implantation or transplantation. The pharmaceutical compositions described herein can be administered to a patient via the arterial, subcutaneous, intradermal, intratumoral, intranodal, intramedullary, intramuscular, by intravenous (i.v.) injection, or intraperitoneal route. The pharmaceutical compositions of the present disclosure may be administered to a patient by intravenous or intratumoral injections. The pharmaceutical compositions can be injected directly into a tumor, lymph node, or site of infection. In some examples, described herein are pharmaceutical compositions for parenteral administration comprising a solution of cells dissolved or suspended in an acceptable carrier, e.g., an aqueous carrier. A variety of aqueous carriers can be used, such as water, buffered water, 0.9% saline, 0.3% glycine, hyaluronic acid, and the like. These pharmaceutical compositions may be sterilized by conventional, well known sterilization techniques, or they may be filter sterilized. The compositions may contain pharmaceutically acceptable auxiliary substances as required to approximate physiological conditions such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like.

The dosage of such treatment to be administered to a subject will vary with the exact nature of the condition being treated and the recipient of the treatment. Dose scaling for human administration (scaling of usages) may be performed according to art-recognized practice. For example, for adult patients, the dose of alemtuzumab is typically in the range of 1 mg to about 100 mg, typically administered daily for a period of 1 to 30 days. A daily dose of 1 to 10 mg/day is preferred, although larger doses of up to 40 mg/day may be used in some cases (described in U.S. Pat. No. 6,120,766 hereby incorporated by reference in its entirety).

It is understood and herein contemplated that the disclosed treatment regimens can used alone or in combination with any anti-cancer therapy known in the art including, but not limited to Abemaciclib, Abiraterone Acetate, Abitrexate® (Methotrexate), ABRAXANE® (Paclitaxel Albumin-stabilized Nanoparticle Formulation), ABVD, ABVE, ABVE-PC, AC, AC-T, ADCETRIS® (Brentuximab Vedotin), ADE, Ado-Trastuzumab Emtansine, Adriamycin® (Doxorubicin Hydrochloride), Afatinib Dimaleate, Afinitor® (Everolimus), Akynzeo® (Netupitant and Palonosetron Hydrochloride), ALDARA® (Imiquimod), Aldesleukin, ALECENSA® (Alectinib), Alectinib, Alemtuzumab, ALIMTA® (Pemetrexed Disodium), ALIQOPA® (Copanlisib Hydrochloride), ALKERAN™ for Injection (Melphalan Hydrochloride), ALKERAN™ Tablets (Melphalan), Aloxi® (Palonosetron Hydrochloride), Alunbrig® (Brigatinib), Ambochlorin® (Chlorambucil), Amboclorin® (Chlorambucil), Amifostine, Aminolevulinic Acid, Anastrozole, Aprepitant, Aredia® (Pamidronate Disodium), Arimidex® (Anastrozole), Aromasin® (Exemestane), Arranon® (Nelarabine), Arsenic Trioxide, Arzerra® (Ofatumumab), Asparaginase Erwinia chrysanthemi, Atezolizumab, Avastin® (Bevacizumab), Avelumab, Axitinib, Azacitidine, Bavencio® (Avelumab), BEACOPP, Becenum® (Carmustine), Beleodaq® (Belinostat), Belinostat, Bendamustine Hydrochloride, BEP, Besponsa® (Inotuzumab Ozogamicin), Bevacizumab, Bexarotene, Bexxar® (Tositumomab and Iodine I 131 Tositumomab), Bicalutamide, BiCNU® (Carmustine), Bleomycin, Blinatumomab, Blincyto® (Blinatumomab), Bortezomib, Bosulif® (Bosutinib), Bosutinib, Brentuximab Vedotin, Brigatinib, BuMel, Busulfan, Busulfex® (Busulfan), Cabazitaxel, Cabometyx® (Cabozantinib-S-Malate), Cabozantinib-S-Malate, CAF, Campath® (Alemtuzumab), Camptosar® (Irinotecan Hydrochloride), Capecitabine, CAPOX, Carac® (Fluorouracil—Topical), Carboplatin, CARBOPLATIN-TAXOL, Carfilzomib, Carmubris® (Carmustine), Carmustine, Carmustine Implant, Casodex® (Bicalutamide), CEM, Ceritinib, Cerubidine® (Daunorubicin Hydrochloride), Cervarix® (Recombinant HPV Bivalent Vaccine), Cetuximab, CEV, Chlorambucil, CHLORAMBUCIL-PREDNISONE, CHOP, Cisplatin, Cladribine, Clafen® (Cyclophosphamide), Clofarabine, Clofarex® (Clofarabine), Clolar® (Clofarabine), CMF, Cobimetinib, Cometriq® (Cabozantinib-S-Malate), Copanlisib Hydrochloride, COPDAC, COPP, COPP-ABV, Cosmegen® (Dactinomycin), Cotellic® (Cobimetinib), Crizotinib, CVP, Cyclophosphamide, Cyfos® (Ifosfamide), Cyramza® (Ramucirumab), Cytarabine, Cytarabine Liposome, Cytosar-U® (Cytarabine), Cytoxan® (Cyclophosphamide), Dabrafenib, Dacarbazine, Dacogen® (Decitabine), Dactinomycin, Daratumumab, Darzalex® (Daratumumab), Dasatinib, Daunorubicin Hydrochloride, Daunorubicin Hydrochloride and Cytarabine Liposome, Decitabine, Defibrotide Sodium, Defitelio® (Defibrotide Sodium), Degarelix, Denileukin Diftitox, Denosumab, DepoCyt® (Cytarabine Liposome), Dexamethasone, Dexrazoxane Hydrochloride, Dinutuximab, Docetaxel, Doxil® (Doxorubicin Hydrochloride Liposome), Doxorubicin Hydrochloride, Doxorubicin Hydrochloride Liposome, Dox-SL® (Doxorubicin Hydrochloride Liposome), DTIC-Dome® (Dacarbazine), Durvalumab, Efudex® (Fluorouracil—Topical), Elitek® (Rasburicase), Ellence® (Epirubicin Hydrochloride), Elotuzumab, Eloxatin® (Oxaliplatin), Eltrombopag Olamine, Emend® (Aprepitant), Empliciti® (Elotuzumab), Enasidenib Mesylate, Enzalutamide, Epirubicin Hydrochloride, EPOCH, Erbitux® (Cetuximab), Eribulin Mesylate, Erivedge® (Vismodegib), Erlotinib Hydrochloride, Erwinaze® (Asparaginase Erwinia chrysanthemi), Ethyol® (Amifostine), Etopophos Etopophos® (Etoposide Phosphate), Etoposide, Etoposide Phosphate, Evacet® (Doxorubicin Hydrochloride Liposome), Everolimus, Evista® (Raloxifene Hydrochloride), Evomela® (Melphalan Hydrochloride), Exemestane, 5-FU® (Fluorouracil Injection), 5-FU® (Fluorouracil—Topical), Fareston® (Toremifene), Farydak® (Panobinostat), Faslodex® (Fulvestrant), FEC, Femara® (Letrozole), Filgrastim, Fludara® (Fludarabine Phosphate), Fludarabine Phosphate, Fluoroplex® (Fluorouracil—Topical), Fluorouracil Injection, Fluorouracil—Topical, Flutamide, Folex® (Methotrexate), Folex PFS® (Methotrexate), FOLFIRI, FOLFIRI-BEVACIZUMAB, FOLFIRI-CETUXIMAB, FOLFIRINOX, FOLFOX, Folotyn® (Pralatrexate), FU-LV, Fulvestrant, Gardasil® (Recombinant HPV Quadrivalent Vaccine), Gardasil 9® (Recombinant HPV Nonavalent Vaccine), Gazyva® (Obinutuzumab), Gefitinib, Gemcitabine Hydrochloride, GEMCITABINE-CISPLATIN, GEMCITABINE-OXALIPLATIN, Gemtuzumab Ozogamicin, Gemzar® (Gemcitabine Hydrochloride), Gilotrif® (Afatinib Dimaleate), Gleevec® (Imatinib Mesylate), Gliadel® (Carmustine Implant), Gliadel wafer® (Carmustine Implant), Glucarpidase, Goserelin Acetate, Halaven® (Eribulin Mesylate), Hemangeol® (Propranolol Hydrochloride), Herceptin® (Trastuzumab), HPV Bivalent Vaccine, Recombinant, HPV Nonavalent Vaccine, Recombinant, HPV Quadrivalent Vaccine, Recombinant, Hycamtin® (Topotecan Hydrochloride), Hydrea® (Hydroxyurea), Hydroxyurea, Hyper-CVAD, Ibrance® (Palbociclib), Ibritumomab Tiuxetan, Ibrutinib, ICE, Iclusig® (Ponatinib Hydrochloride), Idamycin® (Idarubicin Hydrochloride), Idarubicin Hydrochloride, Idelalisib, Idhifa® (Enasidenib Mesylate), Ifex® (Ifosfamide), Ifosfamide, Ifosfamidum® (Ifosfamide), IL-2 (Aldesleukin), Imatinib Mesylate, Imbruvica® (Ibrutinib), Imfinzi® (Durvalumab), Imiquimod, Imlygic® (Talimogene Laherparepvec), Inlyta® (Axitinib), Inotuzumab Ozogamicin, Interferon Alfa-2b, Recombinant, Interleukin-2 (Aldesleukin), Intron A® (Recombinant Interferon Alfa-2b), Iodine I 131 Tositumomab and Tositumomab, Ipilimumab, Iressa® (Gefitinib), Irinotecan Hydrochloride, Irinotecan Hydrochloride Liposome, Istodax® (Romidepsin), Ixabepilone, Ixazomib Citrate, Ixempra® (Ixabepilone), Jakafi® (Ruxolitinib Phosphate), JEB, Jevtana® (Cabazitaxel), Kadcyla® (Ado-Trastuzumab Emtansine), Keoxifene® (Raloxifene Hydrochloride), Kepivance® (Palifermin), Keytruda® (Pembrolizumab), Kisqali® (Ribociclib), Kymriah® (Tisagenlecleucel), Kyprolis® (Carfilzomib), Lanreotide Acetate, Lapatinib Ditosylate, Lartruvo® (Olaratumab), Lenalidomide, Lenvatinib Mesylate, Lenvima® (Lenvatinib Mesylate), Letrozole, Leucovorin Calcium, Leukeran® (Chlorambucil), Leuprolide Acetate, Leustatin® (Cladribine), Levulan® (Aminolevulinic Acid), Linfolizin® (Chlorambucil), LipoDox® (Doxorubicin Hydrochloride Liposome), Lomustine, Lonsurf® (Trifluridine and Tipiracil Hydrochloride), Lupron® (Leuprolide Acetate), Lupron Depot® (Leuprolide Acetate), Lupron Depot-Ped® (Leuprolide Acetate), Lynparza® (Olaparib), Marqibo® (Vincristine Sulfate Liposome), Matulane® (Procarbazine Hydrochloride), Mechlorethamine Hydrochloride, Megestrol Acetate, Mekinist® (Trametinib), Melphalan, Melphalan Hydrochloride, Mercaptopurine, Mesna, Mesnex® (Mesna), Methazolastone® (Temozolomide), Methotrexate, Methotrexate LPF® (Methotrexate), Methylnaltrexone Bromide, Mexate® (Methotrexate), Mexate-AQ® (Methotrexate), Midostaurin, Mitomycin C, Mitoxantrone Hydrochloride, Mitozytrex® (Mitomycin C), MOPP, Mozobil® (Plerixafor), Mustargen® (Mechlorethamine Hydrochloride), Mutamycin® (Mitomycin C), Myleran® (Busulfan), Mylosar® (Azacitidine), Mylotarg® (Gemtuzumab Ozogamicin), Nanoparticle Paclitaxel® (Paclitaxel Albumin-stabilized Nanoparticle Formulation), Navelbine® (Vinorelbine Tartrate), Necitumumab, Nelarabine, Neosar® (Cyclophosphamide), Neratinib Maleate, Nerlynx® (Neratinib Maleate), Netupitant and Palonosetron Hydrochloride, Neulasta® (Pegfilgrastim), Neupogen® (Filgrastim), Nexavar® (Sorafenib Tosylate), Nilandron® (Nilutamide), Nilotinib, Nilutamide, Ninlaro® (Ixazomib Citrate), Niraparib Tosylate Monohydrate, Nivolumab, Nolvadex® (Tamoxifen Citrate), Nplate® (Romiplostim), Obinutuzumab, Odomzo® (Sonidegib), OEPA, Ofatumumab, OFF, Olaparib, Olaratumab, Omacetaxine Mepesuccinate, Oncaspar® (Pegaspargase), Ondansetron Hydrochloride, Onivyde® (Irinotecan Hydrochloride Liposome), Ontak® (Denileukin Diftitox), Opdivo® (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat® (Carboplatin), Paraplatin® (Carboplatin), Pazopanib Hydrochloride, PCV, PEB, Pegaspargase, Pegfilgrastim, Peginterferon Alfa-2b, PEG-Intron® (Peginterferon Alfa-2b), Pembrolizumab, Pemetrexed Disodium, Perjeta® (Pertuzumab), Pertuzumab, Platinol® (Cisplatin), Platinol-AQ® (Cisplatin), Plerixafor, Pomalidomide, Pomalyst® (Pomalidomide), Ponatinib Hydrochloride, Portrazza® (Necitumumab), Pralatrexate, Prednisone, Procarbazine Hydrochloride, Proleukin® (Aldesleukin), Prolia® (Denosumab), Promacta® (Eltrombopag Olamine), Propranolol Hydrochloride, Provenge® (Sipuleucel-T), Purinethol® (Mercaptopurine), Purixan® (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor® (Methylnaltrexone Bromide), R-EPOCH, Revlimid® (Lenalidomide), Rheumatrex® (Methotrexate), Ribociclib, R-ICE, Rituxan® (Rituximab), Rituxan Hycela® (Rituximab and Hyaluronidase Human), Rituximab, Rituximab and, Hyaluronidase Human,, Rolapitant Hydrochloride, Romidepsin, Romiplostim, Rubidomycin® (Daunorubicin Hydrochloride), Rubraca® (Rucaparib Camsylate), Rucaparib Camsylate, Ruxolitinib Phosphate, Rydapt® (Midostaurin), Sclerosol Intrapleural Aerosol (Talc), Siltuximab, Sipuleucel-T, Somatuline Depot® (Lanreotide Acetate), Sonidegib, Sorafenib Tosylate, Sprycel® (Dasatinib), STANFORD V, Sterile Talc Powder (Talc), Steritalc® (Talc), Stivarga® (Regorafenib), Sunitinib Malate, Sutent® (Sunitinib Malate), Sylatron® (Peginterferon Alfa-2b), Sylvant® (Siltuximab), Synribo Synribo® (Omacetaxine Mepesuccinate), Tabloid® (Thioguanine), TAC, Tafinlar® (Dabrafenib), Tagrisso® (Osimertinib), Talc, Talimogene Laherparepvec, Tamoxifen Citrate, Tarabine PFS® (Cytarabine), Tarceva® (Erlotinib Hydrochloride), Targretin® (Bexarotene), Tasigna® (Nilotinib), Taxol® (Paclitaxel), Taxotere® (Docetaxel), Tecentriq® (Atezolizumab), Temodar® (Temozolomide), Temozolomide, Temsirolimus, Thalidomide, Thalomid® (Thalidomide), Thioguanine, Thiotepa, Tisagenlecleucel, Tolak® (Fluorouracil—Topical), Topotecan Hydrochloride, Toremifene, Torisel® (Temsirolimus), Tositumomab and Iodine I 131 Tositumomab, Totect® (Dexrazoxane Hydrochloride), TPF, Trabectedin, Trametinib, Trastuzumab, Treanda® (Bendamustine Hydrochloride), Trifluridine and Tipiracil Hydrochloride, Trisenox® (Arsenic Trioxide), Tykerb® (Lapatinib Ditosylate), Unituxin® (Dinutuximab), Uridine Triacetate, VAC, Vandetanib, VAMP, Varubi® (Rolapitant Hydrochloride), Vectibix® (Panitumumab), VeIP, Velban® (Vinblastine Sulfate), Velcade® (Bortezomib), Velsar® (Vinblastine Sulfate), Vemurafenib, Venclexta® (Venetoclax), Venetoclax, Verzenio® (Abemaciclib), Viadur® (Leuprolide Acetate), Vidaza® (Azacitidine), Vinblastine Sulfate, Vincasar PFS® (Vincristine Sulfate), Vincristine Sulfate, Vincristine Sulfate Liposome, Vinorelbine Tartrate, VIP, Vismodegib, Vistogard® (Uridine Triacetate), Voraxaze® (Glucarpidase), Vorinostat, Votrient® (Pazopanib Hydrochloride), Vyxeos® (Daunorubicin Hydrochloride and Cytarabine Liposome), Wellcovorin® (Leucovorin Calcium), Xalkori® (Crizotinib), Xeloda® (Capecitabine), XELIRI, XELOX, Xgeva® (Denosumab), Xofigo® (Radium 223 Dichloride), Xtandi® (Enzalutamide), Yervoy® (Ipilimumab), Yondelis® (Trabectedin), Zaltrap® (Ziv-Aflibercept), Zarxio® (Filgrastim), Zejula® (Niraparib Tosylate Monohydrate), Zelboraf® (Vemurafenib), Zevalin® (Ibritumomab Tiuxetan), Zinecard® (Dexrazoxane Hydrochloride), Ziv-Aflibercept, Zofran® (Ondansetron Hydrochloride), Zoladex® (Goserelin Acetate), Zoledronic Acid, Zolinza® (Vorinostat), Zometa® (Zoledronic Acid), Zydelig® (Idelalisib), Zykadia® (Ceritinib), and/or Zytiga® (Abiraterone Acetate). The treatment methods can include or further include checkpoint inhibitors including, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B-and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### Kits, Research Reagents, Diagnostics, and Therapeutics

The compositions of the current disclosure can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compositions of the current disclosure, either alone or in combination with other compositions or therapeutics, can be used as tools in differential and/or combinatorial analyses to elucidate expression patterns of a portion or the entire complement of genes expressed within cells and tissues.

In an example, disclosed herein is a kit comprising: a gene modulation reagent configured to inhibit HSD11B1 expression in uterine fibroid cells; a delivery formulation for administering the reagent; and instruction manual for reducing glucocorticoid signaling in fibroid tissue through targeted inhibition of HSD11B1.

In an example, disclosed herein is a gene therapy vector comprising: a promoter operatively linked to a nucleic acid sequence encoding an HSD11B1 inhibitory RNA; a regulatory element that restricts expression to fibroid tissue or uterine smooth muscle cells; and a pharmaceutically acceptable carrier.

In some examples, the inhibitory RNA comprises an shRNA hairpin that targets HSD11B1 transcripts.

In some examples, the regulatory element comprises a tissue selective promoter active in uterine smooth muscle.

As one nonlimiting example, expression patterns within cells or tissues treated with one or more antisense compositions are compared to control cells or tissues not treated with antisense compositions and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compositions which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The compositions of the disclosure are useful for research and diagnostics, because these compositions hybridize to nucleic acids of FKBP5 gene. For example, oligonucleotides that are shown to hybridize with such efficiency and under such conditions as disclosed herein as to be effective FKBP5 gene inhibitors will also be effective primers or probes under conditions favoring gene amplification or detection, respectively. These primers and probes are useful in methods requiring the specific detection of nucleic acid molecules of FKBP5 gene and in the amplification of said nucleic acid molecules for detection or for use in further studies of FKBP5 gene. Hybridization of the antisense oligonucleotides, particularly the primers and probes, of the disclosure with a nucleic acid of FKBP5 gene can be detected by means known in the art. Such means may include conjugation of an enzyme to the oligonucleotide, radiolabelling of the oligonucleotide or any other suitable detection means. Kits using such detection means for detecting the expression of FKBP5 gene in a sample may also be prepared.

The specificity and sensitivity of antisense is also harnessed by those of skill in the art for therapeutic uses. Antisense compositions have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compositions can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of FKBP5 gene is treated by administering antisense compositions in accordance with this disclosure. For example, in one non-limiting example, the methods comprise the step of administering to the animal in need of treatment, a therapeutically effective amount of a FKBP5 gene inhibitor. The FKBP5 gene inhibitors of the current disclosure effectively inhibit the activity of the FKBP51 protein or inhibit the expression of the FKBP51 protein. In one example, the activity or expression of FKBP5 gene in an animal is inhibited by about 10%. Preferably, the activity or expression of FKBP51 protein in an animal is inhibited by about 30%. More preferably, the activity or expression of FKBP 5 gene in an animal is inhibited by 50% or more.

For example, the reduction of the expression of FKBP5 gene may be measured in serum, adipose tissue, liver or any other body fluid, tissue or organ of the animal. Preferably, the cells contained within said fluids, tissues or organs being analyzed contain a nucleic acid molecule of FKBP5 gene protein and/or the FKBP51 protein itself.

The compositions of the disclosure can be utilized in pharmaceutical compositions by adding an effective amount of a FKBP5 modulating agent to a suitable pharmaceutically/therapeutically acceptable diluent or carrier. Use of the compositions and methods of the disclosure may also be useful prophylactically.

The invention will now be further described by way of Examples, which are meant to serve to assist one of ordinary skill in the art in carrying out the invention and are not intended in any way to limit the scope of the invention.

### EXAMPLES

The following examples are set forth below to illustrate the compositions, devices, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1: KBP 5 Knockdown Blunts GR-mediated Transcriptional Activity in Leiomyoma Cells

Uterine leiomyomas are a highly prevalent condition, affecting up to seventy percent of women by menopause, and are associated with pelvic pain, abnormal uterine bleeding, and infertility. Estrogen and progesterone signaling promotes proliferation of leiomyoma cells. Current therapeutic strategies that limit leiomyoma growth and control bleeding primarily rely on sex steroid based approaches, including combined estrogen progesterone oral contraceptives, progestins, and progesterone receptor antagonists. Although newer agents such as GnRH agonists and antagonists effectively alleviate symptoms, these treatments cannot be used long term and must be discontinued in individuals planning pregnancy. The continued need for therapeutic options that do not compromise reproductive goals underscores the importance of understanding the molecular mechanisms governing leiomyoma pathogenesis.

FK506 binding protein 51 is a co chaperone encoded by the FKBP5 gene. This gene contains glucocorticoid and progesterone response elements, although FKBP5 is predominantly induced by glucocorticoids. Glucocorticoid induced FKBP51 interacts with both the glucocorticoid receptor and the progesterone receptor, and inhibits their transcriptional activity by restricting ligand binding. Prior investigations demonstrates that glucocorticoids increase FKBP51 expression in human decidual and endometrial stromal cells, and more recent observations show elevated FKBP51 expression in leiomyomas compared with matched myometrial tissue.

The contribution of glucocorticoid signaling to leiomyoma biology has been examined in several experimental systems. Dexamethasone increases FKBP5 expression, reduces the number of cells in S phase, and suppresses the expression of the estrogen receptor and genes that regulate cell replication in immortalized human uterine leiomyoma cells. In addition, mifepristone, a competitive antagonist of both the progesterone receptor and glucocorticoid receptor with higher binding affinity for the glucocorticoid receptor, inhibits extracellular matrix formation in uterine leiomyoma. Collectively, these findings, along with prior observations, suggest that heightened glucocorticoid and progesterone signaling contributes to leiomyoma progression by increasing FKBP51 expression and promoting extracellular matrix accumulation.

The current invention examines the specific role of FKBP51 in glucocorticoid signaling within leiomyoma cells. Disclosed herein are examples, showing the influence of FKBP51 on global glucocorticoid receptor mediated transcriptional activity in primary leiomyoma cell cultures. This analysis shows that dexamethasone induces HSD11B1, the enzyme responsible for converting cortisone to cortisol, and that FKBP5 silencing reduces dexamethasone mediated induction of HSD11B1. The current results differ from long standing evidence that FKBP51 broadly inhibits glucocorticoid receptor transcriptional activity.

To further investigate the dysregulation of FKBP51 glucocorticoid receptor signaling in leiomyoma, the study evaluates downstream regulation of hydroxysteroid 11 beta dehydrogenase 1 (HSD11β1) in myometrium compared with leiomyoma tissue and identifies increased HSD11B1 expression in leiomyoma samples. These examples support that elevated glucocorticoid signaling in leiomyomatous uteri creates a local positive feedback loop that strengthens FKBP51 glucocorticoid receptor interactions, enhances HSD11B1 expression, and increases local cortisol production. Cortisol then further activates glucocorticoid receptor signaling and amplifies FKBP51 expression. In vitro examples confirm that this positive feedback loop induces a shift from a smooth muscle phenotype to a myofibroblast phenotype, a characteristic feature of leiomyoma cells.

In some examples of the current disclosure, primary leiomyoma cell cultures are used to evaluate how FKBP51 contributes to leiomyoma pathogenesis, including its impact on global GR mediated transcriptional activity. In certain examples, primary leiomyoma cells are transfected with scramble (control) siRNA or FKBP5 siRNA for 48 hours, followed by treatment with either vehicle or 10⁻⁷ M DEX for 24 hours. RNA is then isolated and subjected to whole genome RNA seq analysis.

In some examples, transfection efficiency is assessed by measuring FKBP5 mRNA expression score. FKBP5 siRNA transfected leiomyoma cultures exhibit a 13-fold reduction in FKBP5 mRNA compared with scramble siRNA transfected cultures (0.08±0.04 vs 1.04±0.14; P<0.001). FKBP 51 protein expression are also evaluated by immunoblotting, demonstrating reduced protein levels in FKBP5 siRNA transfected leiomyoma cells relative to scramble siRNA transfected cells.

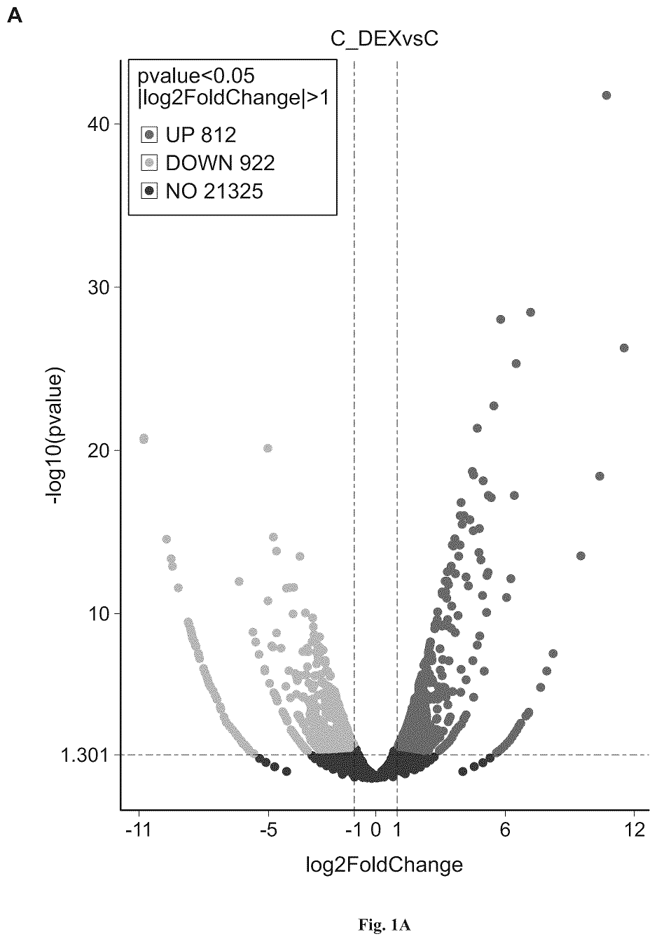

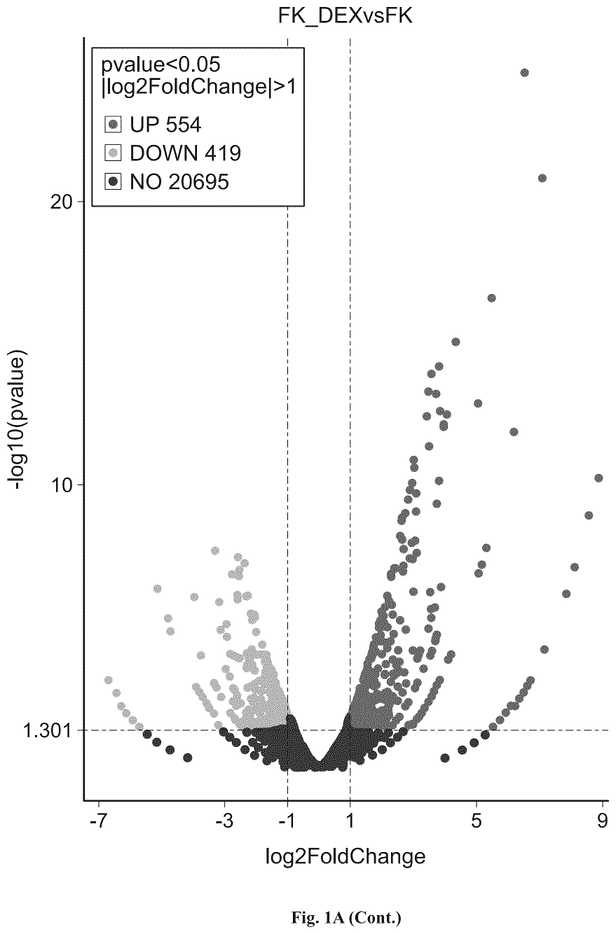

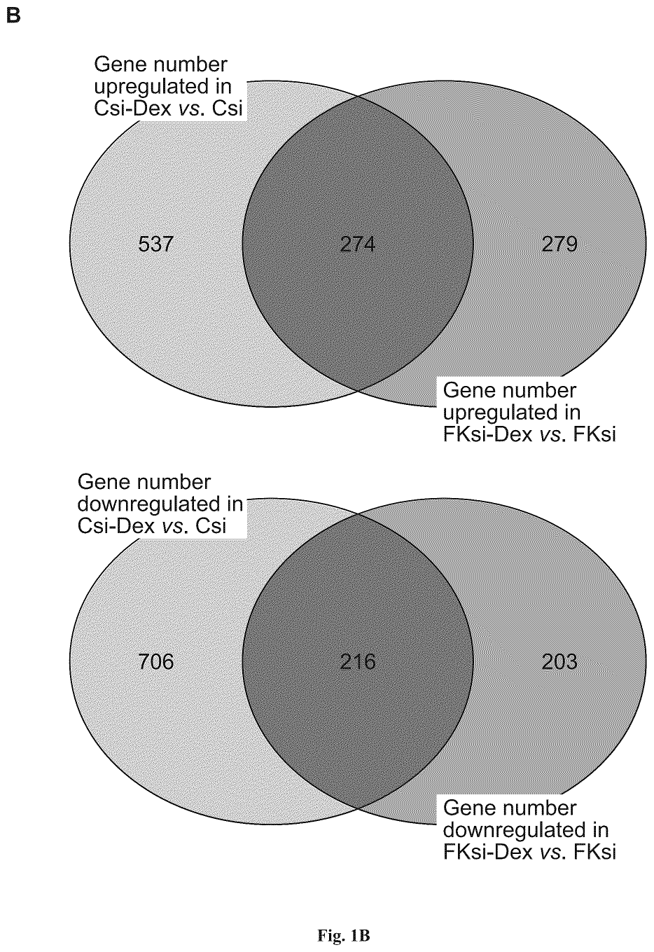

Following RNA sequencing, gene expression profiles are compared between control siRNA transfected cells treated with DEX vs vehicle (C-DEX vs C), as well as between FKBP5 siRNA transfected cells treated with DEX vs vehicle (FK-DEX vs FK). After normalization, genes exhibiting |log2FoldChange|>1 are classified as differentially expressed genes (DEGs) and visualized in Volcano plots (FIG. 1A).

In certain examples, DEX treatment in FKBP5-silenced cells unexpectedly reduces GR mediated transcriptional responses. Specifically, compared with control siRNA transfected cells, FKBP5-silenced cells display fewer DEX-induced upregulated genes (812 vs 554 DEGs) and fewer downregulated genes (922 vs 419 DEGs) (FIG. 1A). Venn diagram assessment further shows that: (1) DEX upregulates 537 genes in control cells, whereas only 279 genes are upregulated in FKBP 5-silenced cells, with 274 genes shared between groups; and (2) DEX downregulates 706 genes in control cells compared with 203 genes in FKBP 5-silenced cells, with 216 genes shared between groups (FIG. 1B).

In some examples, the complete DEG lists corresponding to each Volcano plot are accessible in the NCBI GEO repository under accession number GSE292403.

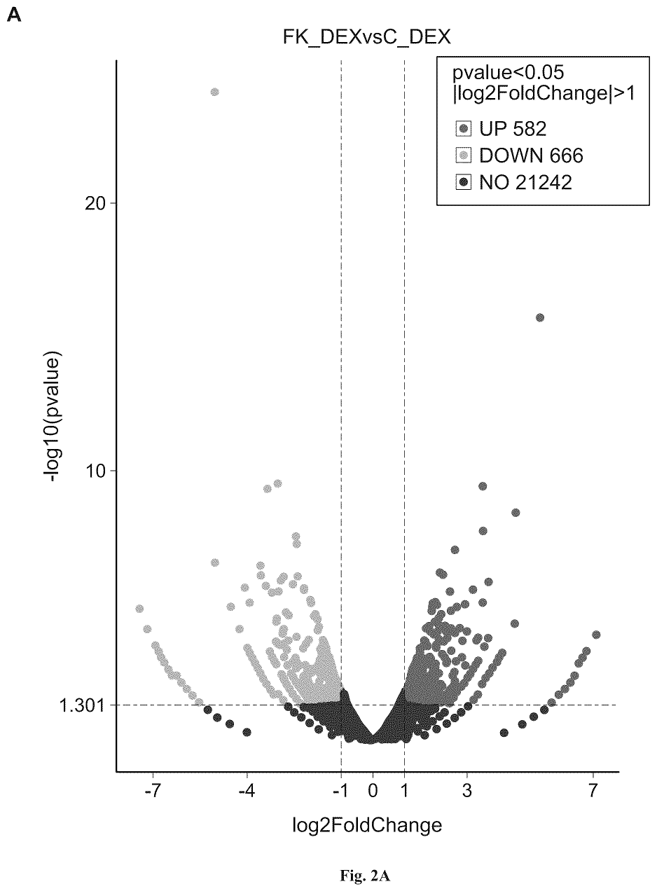

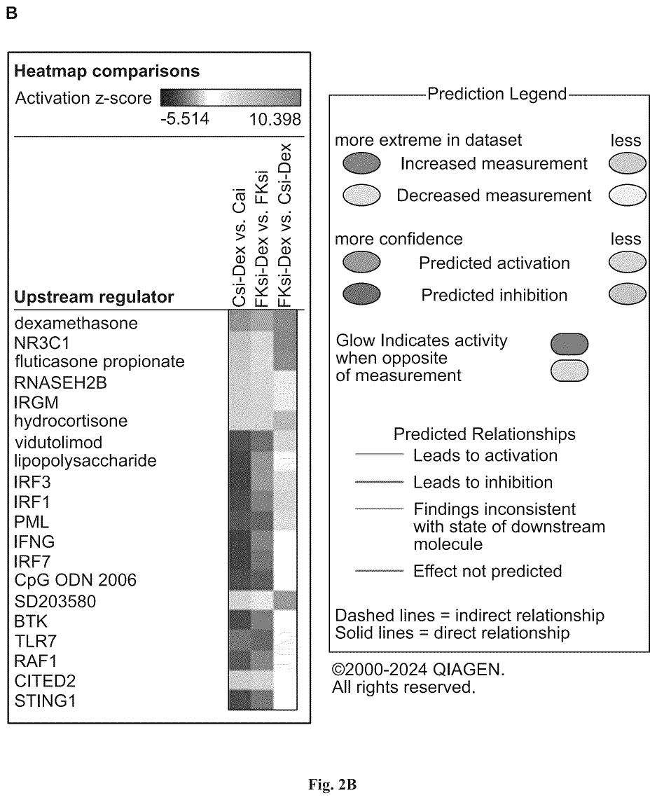

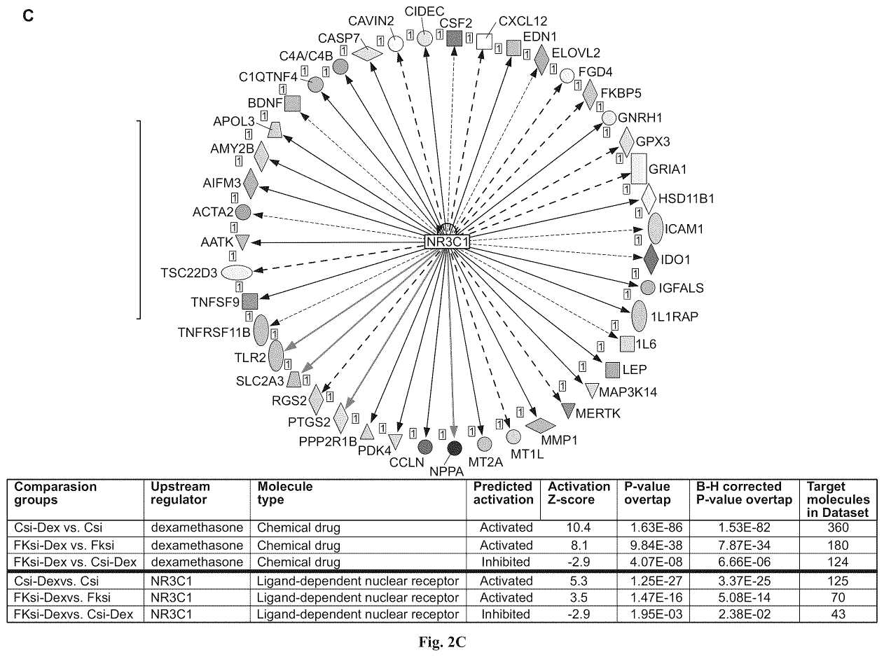

In some examples of the current disclosure, the unexpected findings observed in the comparisons of C-DEX vs C and FK-DEX vs FK prompt further evaluation of DEX-treated FKBP5-silenced cells (FK-DEX) relative to DEX-treated control cells (C-DEX). In certain examples, this comparison identifies a total of 1248 DEGs, consisting of 582 upregulated genes and 666 downregulated genes (FIG. 2A). The complete DEG list is available in the NCBI GEO repository under accession number GSE292403.

In some examples, IPA software (Qiagen) is used to generate heatmap comparisons (FIG. 2B) and to assess activation or inhibition of dexamethasone (DEX) and GR (NR3C1) mediated pathways as upstream regulators of DEGs. This assessment relies on Z score interpretation, where a positive Z score >2 indicates activation and a negative Z score <−2 indicates inhibition, applied across the comparisons C-DEX vs C, FK-DEX vs FK, and FK-DEX vs C-DEX (FIG. 2B).

In certain examples, FKBP5 knockdown reduces the Z score of DEX from 10.4 to 8.1 for its downstream genes and decreases the Z score of NR3C1 from 5.3 to 3.5 for its downstream genes (FIG. 2B). These reduced Z scores indicate that FKBP5 knockdown inhibits DEX and NR3C1 mediated transcriptional activity. This interpretation is further supported by the finding that FKBP5-silenced DEX-treated cultures, when compared to control DEX-treated cultures, exhibit a Z score of −2.9 for both DEX and NR3C1, indicating inhibition of their activity as upstream regulators of DEGs (FIG. 2B).

In some examples, IPA analysis links the inhibition of DEX and NR3C1 activity in the FKD-DEX vs C-DEX comparison to 124 DEGs and 43 DEGs, respectively (FIG. 2B and FIG. 2C). These outcomes differ significantly from earlier studies reporting a consistent inhibitory effect of FKBP51 on GR mediated transcriptional activity.

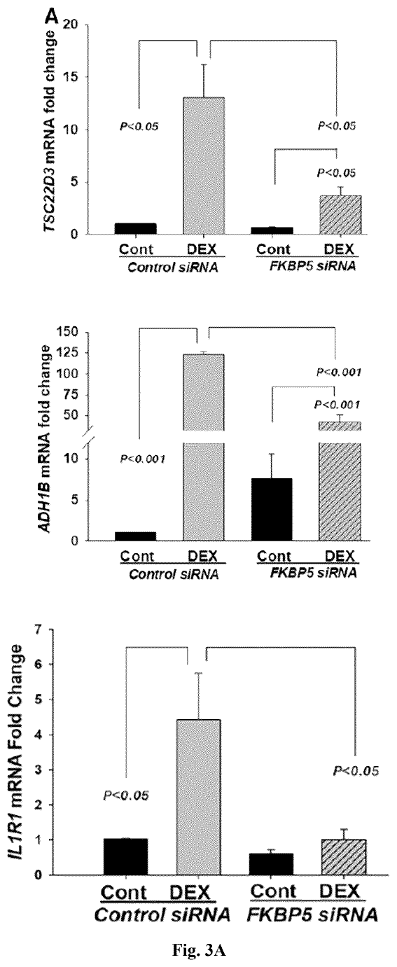

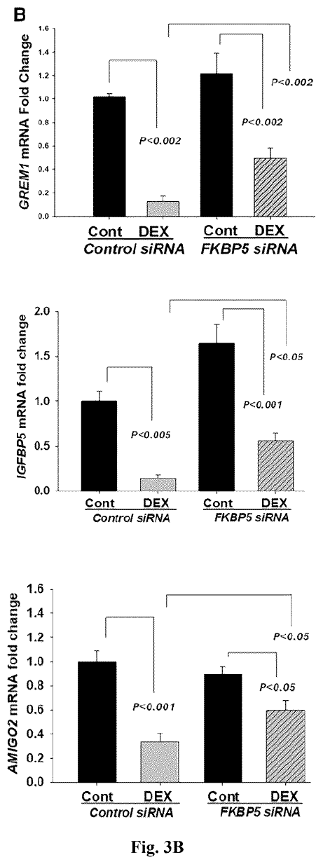

In some examples of the invention, qPCR analysis is performed to validate the RNA seq observations. In certain examples, DEGs known to contain a GR response element are evaluated, including DEX upregulated genes TSC22D3, ADH1B, and IL1R1 (FIG. 3A) and DEX downregulated genes GREM1, IGFBP5, and AMIGO2 (FIG. 3B).

These analyses confirm that FKBP5 silencing in leiomyoma results in dysfunctional regulation of GR mediated transcriptional activity, affecting both DEX-induced and DEX suppressed genes (FIG. 3A and FIG. 3B).

### Example 2: Fkbp 51-dependent Upregulation of HSD11B1 Expression by Dex in Leiomyoma Cells

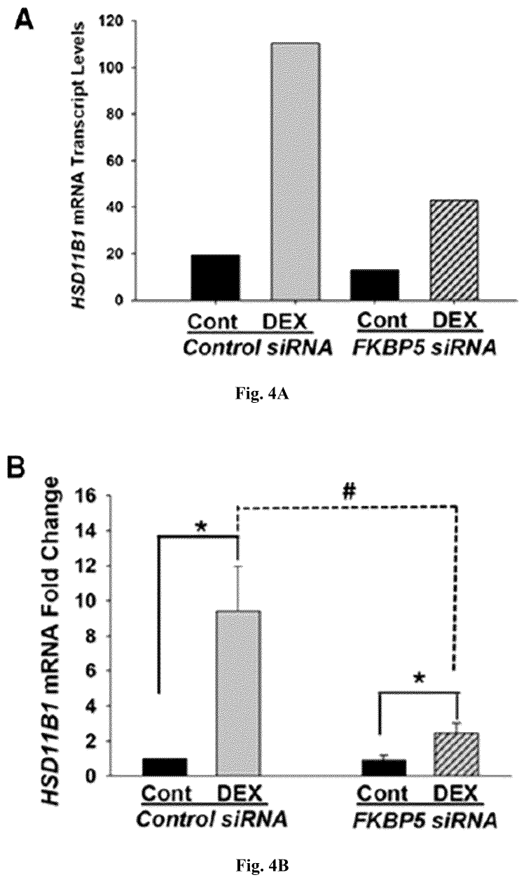

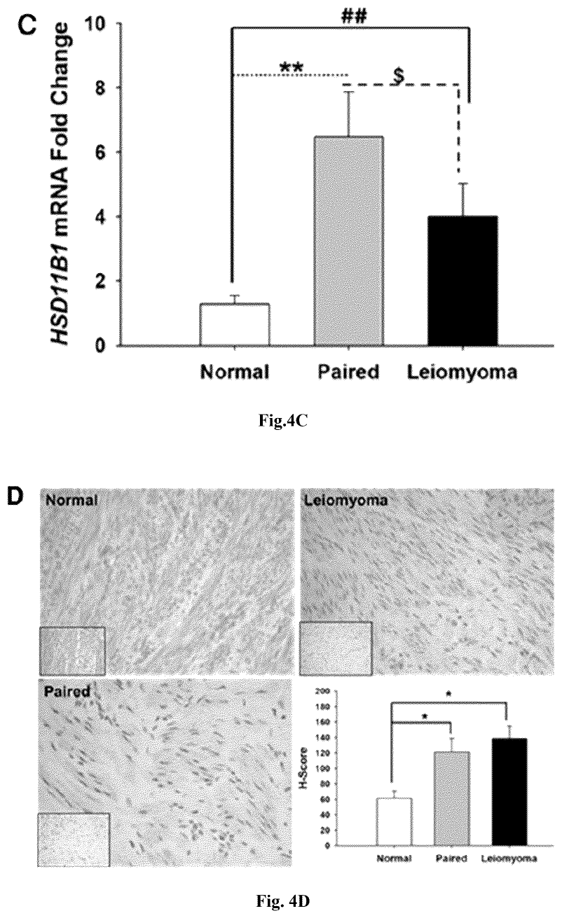

In some examples of the current disclosure, HSD11B1 is identified as a key downstream DEG that responds to FKBP5 silencing. Whole genome sequencing results show a 5.7-fold increase in HSD11B1 expression in leiomyoma cells treated with DEX compared to vehicle controls, whereas FKBP5 knockdown cells exhibit only a 3.3-fold increase under the same DEX-treated conditions (FIG. 4A). The corresponding RNA seq data set is available in the NCBI GEO repository under accession number GSE292403.

In certain examples, qPCR confirmation demonstrates a significant enhancement of HSD11B1 expression in DEX-treated control cells, yielding a 9.4-fold increase relative to control (P<0.05; FIG. 4B). In contrast, FKBP5 siRNA transfected leiomyoma cells treated with DEX show a reduced 2.5-fold increase in HSD11B1 levels compared to control (P<0.05; FIG. 4B). This 2.5-fold induction is significantly lower than the HSD11B1 increase observed in DEX-treated control siRNA transfected cells (P<0.001; FIG. 4B).

In some examples, these findings indicate that FKBP51 contributes to GR mediated transcriptional signaling in leiomyoma cells by promoting DEX-dependent increases in HSD11β1 expression.

### Example 3: in Situ Levels of HSD11β1 Mrna and Protein Expression Are Increased in Leiomyomatous Uteri

In some examples of the current disclosure, HSD11β1 mRNA and protein levels are evaluated in paired myometrial and leiomyoma tissue samples from individuals with leiomyomas, as well as in normal myometrial samples from individuals without leiomyomas. In certain examples, qPCR analysis shows that paired myometrium from uteri with leiomyomas exhibits a 6.5-fold increase in HSD11B1 expression relative to normal myometrium (P=0.002), while leiomyoma samples display a 4-fold increase (P=0.01) (FIG. 4C). The difference between paired myometrium and leiomyoma tissues is also significant (P=0.04).

In some examples, patient samples are categorized by menstrual cycle phase, and tissues collected during the proliferative phase exhibit higher HSD11B1 expression than those collected during the secretory phase across all 3 groups, although this difference does not reach statistical significance due to limited sample power.

In further examples, immunohistochemistry is performed on paired myometrial, leiomyoma, and normal myometrial tissue sections. This analysis confirms that HSD11β1 protein expression parallels the mRNA findings, with paired myometrium and leiomyoma tissues demonstrating significantly higher protein levels than normal myometrium (P<0.05; FIG. 4D).

### Example 4: Dex-induced HSD11B1 Expression by FKBP51-GR Signaling is More Pronounced in Leiomyoma Cells Than Normal Myometrial and Human Endometrial Stromal Cells

In some examples of the current disclosure, the intracellular relationship between FKBP51 and HSD11β1 is evaluated to determine whether it is specific to leiomyoma cells. Cell culture experiments assessing HSD11B1 mRNA expression in leiomyoma cells transfected with control vs FKBP5 siRNA and treated with DEX are repeated in cultured normal myometrial cells and HESCs obtained from uteri without leiomyoma. In certain examples, FKBP5 knockdown efficiency is confirmed by qPCR.

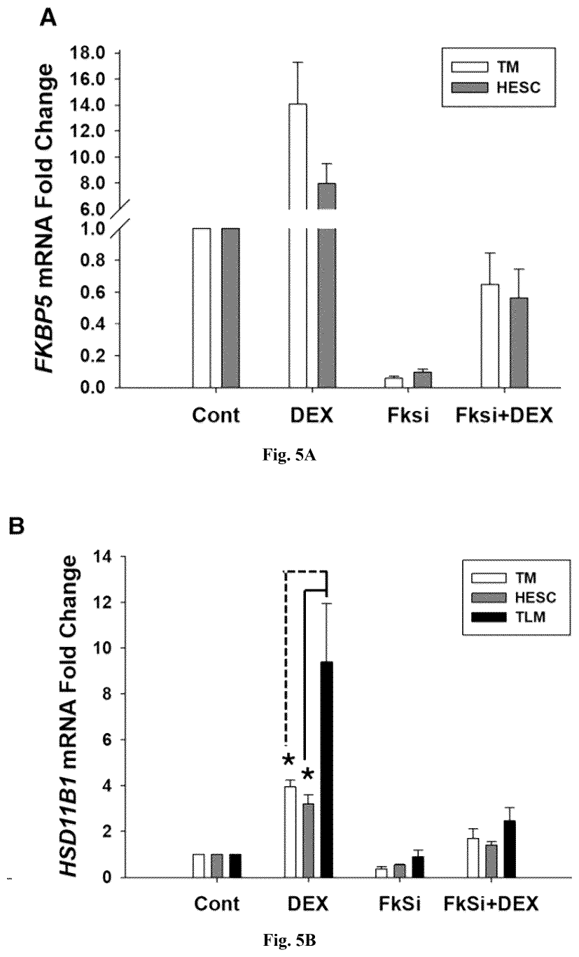

In normal myometrial cultures, FKBP5 siRNA transfected cells exhibit a 17-fold reduction in FKBP5 mRNA levels (0.058±0.01 vs 1.0±0, P<0.001), while HESCs demonstrate a 10.5-fold reduction (0.095±0.02 vs 1.0±0, P<0.001) compared to scramble siRNA transfected cell lines (FIG. 5A).

In some examples, DEX increases HSD11B1 mRNA levels by 4-fold in normal myometrial cells and by 3.2-fold in HESCs. These increases are significantly lower than the 9.4-fold upregulation detected in leiomyoma cells (P<0.05; FIG. 5B).

Following FKBP5 knockdown, DEX still increases HSD11B1 levels in all 3 cell types. However, these increases are notably reduced compared to control siRNA transfected counterparts, showing 1.7-fold in normal myometrial cells (P<0.05), 1.4-fold in HESCs (P<0.05), and 2.5-fold in leiomyoma cells (P<0.05). In certain examples, these post knockdown increases in HSD11B1 are not significantly different among the 3 cell types (P=0.248; FIG. 5B).

In some examples, these findings indicate that DEX-induced GR signaling increases HSD11B1 expression in a FKBP51-dependent manner across different types of uterine cells, with leiomyoma cells exhibiting a much greater response in the presence of FKBP51.

### Example 5: Enhanced FKBP 51-glucocorticoid Signaling Induces Leiomyoma Cells Toward a Myofibroblast Phenotype

In some examples of the current disclosure, the contribution of elevated FKBP51-GR signaling to leiomyoma pathogenesis is evaluated by comparing transcriptional changes in genes encoding extracellular matrix (ECM) proteins and genes associated with smooth muscle cell proteins. In certain examples, expression of ECM related genes including LAMA2, LAMB1, and FN1, and smooth muscle associated genes including CNN1, MYH9, MYH10, and ACTA2 is assessed in cultured leiomyoma cells transfected with control vs FKBP5 siRNA and treated with DEX.

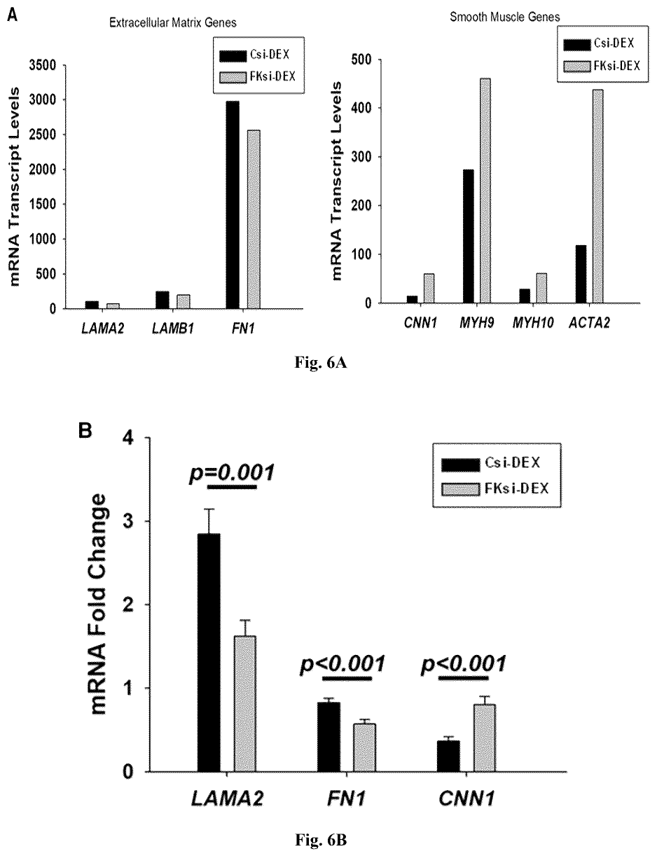

In some examples, RNA sequencing data demonstrate that in the FKD-DEX vs C-DEX comparison, transcription levels of ECM genes are reduced, while genes encoding proteins characteristic of smooth muscle cells are increased (FIG. 6A). The raw sequencing data set is available in the NCBI GEO repository under accession number GSE292403.

To confirm these observations, qPCR analysis is performed to evaluate expression levels of LAMA2, FN1, and CNN1. In certain examples, DEX-treated FKBP5-silenced cells display significantly lower levels of LAMA2 (P=0.001) and FN1 (P=0.001) relative to DEX-treated control cells. Conversely, CNN1 levels are significantly higher in DEX-treated FKBP5-silenced cells compared with DEX-treated control cells (P<0.001; FIG. 6B).

In some examples, these findings suggest that increased FKBP51-GR signaling contributes to a phenotypic shift in leiomyoma cells, promoting the transition from a smooth muscle phenotype to a myofibroblast phenotype.

### Example 6: FKBP 51-GR Signaling Interactions Contribute to the Pathogenesis of Leiomyoma

In some examples of the current disclosure, whole genome RNA seq analysis in primary leiomyoma cell cultures demonstrates that FKBP51 amplifies GR mediated transcriptional activity, resulting in both upregulated and downregulated gene expression. These observations differ from earlier reports suggesting that FKBP51 broadly inhibits GR mediated transcriptional responses. In certain examples, HSD11B1 is identified as a key downstream mediator of this enhanced FKBP51-GR signaling. Both in vitro and in situ analyses confirm increased HSD11β1 expression in leiomyomatous uteri compared with normal myometrium.

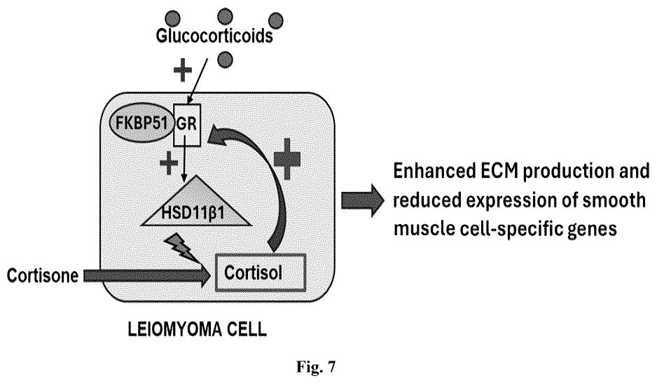

In some examples, increased FKBP51-GR signaling in leiomyoma cells is shown to induce a transition from a smooth muscle phenotype to a myofibroblast phenotype. This finding aligns with widely documented observations of increased extracellular matrix deposition in uterine leiomyoma. The current disclosure suggests that elevated GR signaling in leiomyomatous uteri forms a local positive pathological feedback loop in which increased FKBP51 levels enhance HSD11β1 expression, leading to increased production of active cortisol. The elevated cortisol then sustains GR activation, further increases FKBP51 levels, induces expression of ECM genes, and suppresses genes encoding smooth muscle structural proteins (FIG. 7).

In certain examples, the tissue specific nature of FKBP51 GR interactions is considered. Prior work associating FKBP51 with inhibition of GR signaling has focused largely on neuronal tissues. Additional studies from the inventors establish inhibitory roles for FKBP51 in PR and GR signaling in decidual and endometrial stromal cells. The present findings of increased FKBP51-GR signaling through upregulation of HSD11β1 in leiomyoma cells indicate that this effect may be specific to leiomyoma or uterine tissue. Further analysis in other tissue types, including neuronal cells, may clarify whether FKBP51 GR induction of HSD11β1 occurs beyond uterine environments and whether such pathways could be leveraged for alternative therapeutic applications.

In some examples, the relative roles of glucocorticoid metabolizing enzymes are evaluated. HSD11β1 is not the sole regulator of intracellular glucocorticoid activity; HSD11β2 also contributes by converting active cortisol to inactive cortisone. The balance between these enzymes determines the magnitude of GR signaling and varies by tissue and cell type. In certain examples from leiomyoma cell culture RNA seq data, HSD11B2 transcript levels are extremely low, at <0.1 fragments per kilobase of transcript per million mapped reads, and remain extremely low after FKBP5 silencing. Although DEX treatment increases HSD11B2 levels 4.6-fold, expression remains <0.1 fragments per kilobase per million mapped reads. Additional prior analyses show no differences in FKBP52, a positive regulator of GR signaling, in leiomyoma vs paired myometrium. Because DEX is a synthetic glucocorticoid that does not require HSD11β1 or HSD11β2 for activation or inactivation, these findings suggest that the upregulation of HSD11β1 occurs through FKBP51 elevation either directly or indirectly via an unidentified mediator.

In some examples, potential involvement of mediator complex components is considered. Prior work has shown differential GR binding and GR target gene regulation involving mediator of RNA polymerase II subunits, including MED1 and MED14. The mediator subunit MED12 is frequently mutated in leiomyoma, with mutations detected in 55.8% of tumors in recent analyses. These mutations are associated with increased AKT signaling, reduced cyclin C CDK8/19 kinase activity, and increased myometrial cell growth and proliferation. Because MED12 mutations alter mediator complex function and transcriptional regulation, a potential interaction between MED12, GR signaling, and FKBP51 may be relevant and warrants future exploration.

In some examples, these findings provide a basis for new therapeutic approaches. Current leiomyoma treatments primarily target estrogen and progesterone pathways or block GnRH signaling. The present disclosure suggests that targeting FKBP51-mediated GR signaling, or inhibiting HSD11β1 expression or activity, may interrupt the FKBP51-GR-HSD11β1 cycle in leiomyomatous uteri. Such intervention could reduce ECM protein production and decrease the progression or formation of leiomyomas (FIG. 7). This approach may be particularly beneficial for individuals who cannot use hormone-based therapies or who wish to maintain fertility.

### MATERIALS AND METHODS

### Collection of Human Specimens

In some examples of the current disclosure, uterine specimens are collected following patient consent and approval by an institutional review board. Paired leiomyoma and adjacent normal myometrial tissues are obtained at least 1 cm apart, consistent with procedures described in prior work. Normal myometrial specimens are isolated from premenopausal individuals without leiomyoma who undergo hysterectomy for conditions including endometriosis, pelvic pain, benign adnexal masses, or cervical dysplasia, and who have not received hormonal medications within 3 months before surgery. In certain examples, endometrial samples are obtained either via a Pipelle suction curette before hysterectomy or from endometrial scrapings collected immediately after hysterectomy.

### Cell Cultures

In some examples, frozen aliquots of previously isolated, characterized primary leiomyoma cells are cultured in basal media consisting of phenol red free DMEM F12 supplemented with 10% FBS and 1% antibiotic and antimycotic solution. These cells are expanded until confluency.

Normal myometrial tissue samples were collected in Hanks'balanced salt solution (Thermo Fisher Scientific) within 1 hour of surgery. The myometrial tissues were minced and digested in a buffer containing 0.2 mg/mL deoxyribonuclease I DNase I (Roche Diagnostics, Indianapolis, IN) and 5 mg/mL collagenase type II (5 mg/mL; Thermo Fisher Scientific) for 45 minutes at 37° C. with gentle agitation. The digested tissue pieces were then plated into 10-cm tissue culture dishes and cultured in basal media supplemented with 10 ng/mL epidermal growth factor (R&D Systems, Minneapolis, MN) until the primary outgrowing cells reached approximately 70% confluency. For subculturing, the growing cells were trypsinized using 0.25% trypsin and 0.05% EDTA. The smooth muscle like properties of these cultured myometrial cells are previously validated.

In additional examples, endometrial tissues are collected in Hanks balanced salt solution within one hour of surgery for the isolation of human endometrial stromal cells. Minced tissue fragments are digested in DMEM F12 containing collagenase B (1 mg/mL, 15 U/mg (Roche), deoxyribonuclease I (0.1 mg/mL, 1500 U/mg; Roche), penicillin (200 U/mL), and streptomycin (200 mg/mL) for 30 minutes at 37° C. with gentle agitation. The dispersed endometrial cells were separated by filtration through a 70-μm diameter cell strainer and transferred to basal media for continued culture until confluence.

### Silencing of FKBP5 by Sirna and Culture Treatments

In some examples, leiomyoma, myometrial, and endometrial stromal cell cultures, each seeded at 1.5×10⁵ cells per well, are incubated in basal media for twenty four hours in six well plates. The cultures are then transfected with twenty nM nonspecific control siRNA or FKBP5 specific siRNA using a lipofectamine based transfection reagent in OptiMEM serum reduced medium. After forty eight hours, the cells are treated with either vehicle medium containing DMEM F12 with 2% FBS or dexamethasone (DEX) at 10⁻⁷ M for twenty four hours. Following treatment, cells are washed with ice cold PBS and stored at a temperature of −80° C. for RNA isolation. In some examples, silencing efficiency of FKBP5 at both the mRNA and protein levels is confirmed by quantitative PCR and immunoblotting.

### RNA Isolation and Reverse Transcription

In some examples of the current disclosure, total RNA is isolated from: (1) leiomyoma and paired myometrial tissues from the proliferative (n=9) and secretory (n=8) phases; (2) normal myometrial tissues from the proliferative (n=5) and secretory (n=11) phases; and (3) cultured leiomyoma (n=4), normal myometrial (n=4), and human endometrial stromal cells (n=4) previously transfected with control or FKBP5 siRNA±DEX. In certain examples, isolation is performed using the miRNeasy Mini Kit (Qiagen Inc, Germantown, MD). In some examples, reverse transcription is performed using the Qiagen Omniscript RT kit.

### RNA Sequencing and Analysis

In some examples of the invention, RNA samples from control or FKBP5 siRNA transfected ±10⁻⁷ M DEX-treated primary leiomyoma cultures (n=4) are equally pooled and processed for whole genome RNA sequencing using the NovaSeq 6000 platform. RNA samples used for RNA seq exhibit an RNA Integrity Number (RIN) >8. Following normalization, genes with a-fold change >1.5 and P<0.05 are identified as differentially expressed in DEX-treated control or FKBP5-silenced cells compared with vehicle treated control or FKBP5-silenced cells. In some examples, upstream regulator analysis, heat map generation, and volcano plot generation are completed using Ingenuity Pathway Analysis (IPA) software. In certain examples, qPCR is performed in these cultures (n=4) to confirm differentially expressed genes.

### Real Time Quantitative Pcr

In some examples, qPCR is conducted using gene specific TaqMan gene expression assays. The full name and Taq-Man Probe Assay IDs for each target gene include: FK506-binding protein 5 (FKBP 5)-Hs01561006_m1; Hydroxysteroid 11-β dehydrogenase 1 (HSD11B1)-Hs01547870_m1; IL-1 receptor type 1 (IL1R1)-Hs00991002_m1; TSC22 domain family member 3 (TSC22D3)-Hs00608272_m1; alcohol dehydrogenase 1B, β polypeptide (ADH1B)-Hs00605175_m1; Gremlin 1, DAN family BMP antagonist (GREM1)-Hs00171951_m1; IGF-binding protein 5 (IGFBP5)-Hs00181213_m1; adhesion molecule with Ig-like domain 2 (AMIGO2)-Hs05001325_ s1; laminin subunit alpha 2 (LAMA2)-Hs00166308_m1; fibronectin 1 (FN1)-Hs00365052_m1; calponin 1 (CNN1)-Hs00959434_m1; actin β (ACTB)-Hs99999903_m1; and glyceraldehyde-3-phosphate dehydrogenase (GAPDH)-Hs99999905_m1. In some examples, all samples are run in duplicate. ACTB and GAPDH serve as endogenous controls. Relative-fold change is calculated using the 2^{-ΔΔCT} method.

### Immunohistochemistry

In the current disclosure, immunostaining is performed to detect endogenous HSD11β1 levels in leiomyoma (n=6), paired myometrial (n=6), and normal myometrial tissues (n=6). These specimens include both mid proliferative (n=3) and mid secretory (n=3) phase tissues.

Five μm serial sections of 4% paraformaldehyde fixed, paraffin embedded tissue are deparaffinized and rehydrated, then incubated in 3% hydrogen peroxide to block endogenous peroxidase activity. Antigen retrieval is performed by boiling in Tris EDTA buffer (10 mM Tris base, 1 mM EDTA; pH 9.0) for 20 minutes. After washing with TBS, slides are incubated with 5% normal goat serum (Vector Labs, Burlingame, CA) for 30 minutes at room temperature, followed by overnight incubation at 4° C. with rabbit polyclonal antibody against HSD11β1 (1:100, Atlas Antibodies Cat# HPA 047729, RRID:AB_2680135) in 2.5% goat serum.

As a negative control, sections are incubated with nonspecific rabbit IgG. Slides are washed in TBS with 1% Tween 20, then incubated with biotinylated goat anti rabbit secondary IgG (1:400; Vector Labs) for 30 minutes. Antigen antibody complexes are visualized following incubation with streptavidin biotin peroxidase complex (Elite ABC kit; Vector Labs) for 30 minutes and 3,3 diaminobenzidine for 5 minutes at room temperature. Nuclei are counterstained with hematoxylin. Slides are mounted using an aqueous based mounting medium.

### Statistical Analysis

In some examples, statistical analysis is conducted using SigmaStat version 11.0 (Systat Software, Inc., San Jose, CA). Pair wise multiple group comparisons are performed by 1 way ANOVA followed by Tukey post hoc test for parametric distributions or Student Newman Keuls for nonparametric distributions. For comparison of 2 groups, either a t test or Mann Whitney Wilcoxon rank sum test is applied depending on distribution type. Statistical significance is defined as P<0.05.

Those skilled in the art will appreciate that numerous changes and modifications can be made to the preferred examples of the invention and that such changes and modifications can be made without departing from the spirit of the invention. It is, therefore, intended that the appended claims cover all such equivalent variations as fall within the true spirit and scope of the invention.

Throughout this application, various publications are referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon.

What is claimed is:

1. A method of treating a uterine disorder in a subject, comprising:
determining an expression score of one or more biomarkers in a biological sample obtained from a subject relative to a reference control,
wherein the one or more biomarkers comprise one or more RNAs, or an expression product thereof, of genes selected from the group consisting of TSC22D3, ADH1B, IL1R1, HSD11B1, GREM1, IGFBP5, AMIGO2, and FKBP5;
determining a cumulative expression score from the one or more expression scores of the one or more biomarkers; and
administering to the subject a therapeutically effective amount of an FKBP5 modulating agent if the cumulative expression score is lower than a threshold.

2. The method of claim 1, wherein the expression score greater than 1 indicates upregulation relative to the reference control and an expression score less than 1 indicates downregulation relative to the reference control.

3. The method of claim 1, wherein the FKBP5 modulating agent comprises an FKBP5 small interfering RNA.

4. The method of claim 1, wherein the FKBP5 modulating agent comprises a small molecule inhibitor (siRNA) of FKBP51 protein activity.

5. The method of claim 1, wherein the FKBP5 modulating agent comprises an inhibitor of HSD11B1.

6. The method of claim 1, wherein the biological sample comprises leiomyoma tissue, paired myometrial tissue, myometrial tissue, endometrial stromal cells, or any combination thereof.

7. The method of claim 1, wherein the uterine disorders comprise uterine leiomyoma, uterine fibroids, abnormal uterine bleeding, myometrial hyperplasia, progesterone resistant uterine disorder, estrogen responsive uterine disorder, endometriosis, pelvic pain, benign adnexal mass, adenomyosis, uterine cancer, polycystic ovary syndrome (PCOS) or cervical dysplasia.

8. A method of treating a uterine disorder in a subject, comprising:
determining an expression score of a first group of one or more biomarkers, a second group of one or more biomarkers, a third group of one or more biomarkers, and a fourth group of one or more biomarkers in a biological sample obtained from a subject relative to a reference control,
wherein the first group of one or more biomarkers comprise one or more RNAs, or an expression product thereof, of genes selected from the group consisting of CNN1, MYH9, MYH10, and ACTA2, the second group of one or more biomarkers comprise one or more RNAs, or an expression product thereof, of the genes selected from the group consisting of GREM1, IGFBP5, and AMIGO2, the third group of one or more biomarkers comprise one or more RNAs, or an expression product thereof, of the genes selected from the group consisting of LAMA2, LAMB1, and FN1, and the fourth group of one or more biomarkers comprise one or more RNAs, or an expression product thereof, of the genes selected from the group consisting of TSC22D3, ADH1B, IL1R1, and HSD11B1;
determining a first expression score by integrating the expression scores of the one or more biomarkers in the first group, a second expression score by integrating the expression score of the one or more biomarkers in the second group, a third expression score by integrating the expression score of the one or more biomarkers in the third group, and a fourth expression score by integrating the expression score of the one or more biomarkers in the fourth group; and
administering to the subject a therapeutically effective amount of an FKBP5 modulating agent if the first expression score is lower than a first threshold, the second expression score is lower than a second threshold, the third expression score is higher than a third threshold, and the fourth expression score is higher than a fourth threshold.

9. The method of claim 8, comprising administering to the subject a therapeutically effective amount of the FKBP5 modulating agent if the first expression score is lower than 0.5, the second expression score is lower than 0.5, the third expression score is higher than 2, and the fourth expression score is higher than 2.

10. The method of claim 8, wherein the biological sample from the subject has a higher expression of FKBP51 or HSD11β1 protein as compared to the reference control.

11. The method of claim 8, wherein the FKBP5 modulating agent comprises an FKBP5 small interfering RNA (siRNA).

12. The method of claim 8, wherein the FKBP5 modulating agent comprises a small molecule inhibitor of FKBP51 protein activity.

13. The method of claim 8, wherein the FKBP5 modulating agent comprises an inhibitor of HSD11B1.

14. The method of claim 8, wherein the biological sample comprises leiomyoma tissue, paired myometrial tissue, myometrial tissue, endometrial stromal cells, or any combination thereof.

15. The method of claim 8, wherein the uterine disorders comprise uterine leiomyoma, uterine fibroids, abnormal uterine bleeding, myometrial hyperplasia, progesterone resistant uterine disorder, estrogen responsive uterine disorder, endometriosis, pelvic pain, benign adnexal mass, adenomyosis, uterine cancer, polycystic ovary syndrome (PCOS) or cervical dysplasia.

16. A pharmaceutical composition, comprising: a FKBP5 modulating agent; and a therapeutically acceptable carrier.

17. The composition of claim 16, wherein the FKBP5 modulating agent comprises an FKBP5 small interfering RNA (siRNA).

18. The composition of claim 17, wherein the FKBP5 siRNA comprises 8-30 nucleotides targeting a nucleic acid molecule encoding FKBP5 gene.

19. The composition of claim 16, wherein the FKBP5 modulating agent comprises a small molecule inhibitor of FKBP51 protein activity.

20. The composition of claim 16, wherein the FKBP5 modulating agent comprises an inhibitor of HSD11B1.

21. The composition of claim 16, wherein the therapeutically acceptable carrier comprises polymer micelle, liposome, emulsion, microsphere, or nanosphere.

22. The composition of claim 16, wherein the FKBP5 modulating agent is an antisense oligonucleotide, wherein the antisense oligonucleotide is a DNA oligonucleotide, an RNA oligonucleotide, or a chimeric oligonucleotide.

23. The composition of claim 22, wherein the antisense oligonucleotide hybridizes with an FKBP5 RNA to form an oligonucleotide-RNA duplex.

24. The composition of claim 16, wherein the FKBP 5 modulating agent shares at least 70% complementarity with a nucleic acid molecule encoding FKBP5 gene.
